(19) United States
(12) Patent Application Publication
(10) Pub. No.: US 2024/0301474 A1
(43) Pub. Date: Sep. 12, 2024

(54) NUCLEASES FOR SIGNAL AMPLIFICATION

(71) Applicant: Amazon Technologies, Inc., Reno, NV (US)

(72) Inventors: Carla Alejandra GIMENEZ, Buenos Aires (AR); Federico Alberto PEREYRA BONNET, Buenos Aires (AR); Ailin SVAGZDYS ABAD, Buenos Aires (AR)

(21) Appl. No.: 17/772,960

(22) PCT Filed: Apr. 14, 2022
(86) PCT No.: PCT/US2022/024914

§ 371 Date: Apr. 28, 2022

Related U.S. Application Data
(60) Provisional application No. 63/175,236, filed on Apr. 15, 2021.

(51) Int. Cl. C12Q 1/682; C12N 9/22; C12N 15/11; C12Q 1/70

(52) U.S. Cl. CPC C12Q 1/682; C12N 9/22; C12N 15/11; C12Q 1/70; C12N 2310/20

(57) ABSTRACT

Provided herein are methods that utilize a CRISPR/Cas complex having collateral activity, one or more nucleases, one or more oligonucleotides and a fluorescent reporter. The methods disclosed herein can amplify a fluorescent signal when a target nucleic acid is present in a sample.

### RELATED APPLICATIONS

This application claims the benefit U.S. Provisional Patent Application No. 63/175,236, filed Apr. 15, 2021, the entire contents of which are hereby incorporated herein by reference.

### REFERENCE TO A SEQUENCE LISTING

This application contains a Sequence Listing in computer readable form. The computer readable form is incorporated herein by reference. Said ASCII copy, created on Apr. 6, 2022, is named 146401_091762_SL.txt and is 74,633 bytes in size.

### BACKGROUND

The ability to measure nucleic acids with rapid, highly sensitive, specific, and cost-effective methods is crucial for a number of applications in human health and biotechnology, such as identification and detection of infectious diseases, agricultural pathogens, or circulating DNA or RNA associated with disease.

While methods have been developed for sensing nucleic acids, they all suffer from drawbacks, for example they lack sensitivity and specificity to detect nucleic acids at low concentrations, are expensive, time-consuming or too complex to use outside of laboratories. Polymerase chain reaction (PCR) is one of the most commonly used methods for detecting nucleic acids. However, PCR is expensive, requires specialized and complex instrumentation, limiting usability to specially trained personnel. Other approaches such as isothermal nucleic acid amplification is faster than PCR and can be operated at a constant temperature, eliminating the need for sophisticated equipment like thermocyclers, but have limited applications due to low sensitivity and specificity (Zanoli et al., Biosensors., 3:18-43 (2013)). As such, isothermal nucleic acid amplification cannot typically discriminate between single-base pair differences in target sequences, a distinction that can have important consequences for pathogenicity.

Two technologies using CRISPR-based gene editing have been developed to detect nucleic acids, SHERLOCK (specific high-sensitivity enzymatic reporter unlocking) (Kellner et al., Nat. Protoc., 14(1):2986-3012 (2019)) and DETECTR (Broughton et al., Nature Biotechnology, 38, 870-874 (2020)). However, each of these technologies requires pre-amplifying RNA (or DNA with a reverse transcriptase) using a conventional technique, such as isothermal amplification.

Thus, there is a need for new methods that are suitable for rapidly identifying and sensing nucleic acids that can be used for versatile point-of-care applications with high-specificity and sensitivity, and are cost-effective.

### SUMMARY

Cas effector nucleases from particular types of CRISPR/Cas complexes have been found to exhibit target-dependent promiscuous nuclease RNase activity (e.g., RNase activity and DNase activity), leading to trans cleavage of bystander RNA/DNA molecules (also referred to as collateral activity). The inventors have developed novel methods that take advantage of CRISPR/Cas complexes that exhibit collateral activity. The novel methods can be used to rapidly detect nucleic acids with high sensitivity in a single assay. The methods disclosed herein do not required pre-amplification of nucleic acids like other tools. The method has the capability of amplifying a reporter signal through the use of one or more nucleases and one or more oligonucleotides. As a result, the method can easily distinguish similar sequences (e.g., similar viruses).

This disclosure relates to novel methods using a CRISPR/Cas system that can be used to identify and detect a target nucleic acid in a sample. Specifically, the methods disclosed herein comprise providing to a sample (i) a CRISPR/Cas complex comprising an effector nuclease and a guide RNA encoding a nucleic acid that hybridizes to a target nucleic acid, (ii) one or more nucleases, (iii) one or more oligonucleotides, and (iv) a fluorescence reporter. Preferably, the one or more nucleases is not the same as the effector nuclease. The method can also include measuring a fluorescence signal emitted from the fluorescence reporter. The presence of a target nucleic acid can be detected by presence of a fluorescence signal. The methods disclosed herein are particularly useful for detecting viral nucleic acids.

Without being bound by theory or mechanism, the inventors believe that when a target nucleic acid is present in a sample the effector nuclease (e.g., a Cas protein) is directed to a target nucleic acid sequence by the guide RNA. The guide RNA interacts with the Cas protein and encodes a nucleic acid that hybridizes to a target nucleic acid. Once directed to the target nucleic acid, the Cas protein is capable of cleaving the target nucleic acid. When the CRISPR/Cas complex has been activated by cleaving the target nucleic acids, the CRISPR/Cas complex cleaves one or more oligonucleotides through its collateral activity. The oligonucleotides act as a second messenger and activate one or more nucleases. The one or more nucleases then cleave the fluorescence reporter and amplifies the fluorescence signal. The presence of a fluorescence signal indicates the presence of a target nucleic acid. The absence of a fluorescence signal indicates the absence of a target nucleic acid.

Also described herein are methods for identifying a subject having a disease. The method for identifying a subject having a disease can comprise providing to a sample (i) a CRISPR/Cas complex comprising an effector nuclease and a guide RNA encoding a nucleic acid that hybridizes to a target nucleic acid, (ii) one or more nucleases, (ii) one or more oligonucleotides, and (iv) a fluorescence reporter, and measuring a fluorescence signal emitted from the fluorescence reporter. Preferably, the one or more nucleases is not the same as the effector nuclease. The presence of the fluorescence signal indicates the presence of disease.

In addition, the disclosure provides kits comprising the reagents used in the methods disclosed herein for identifying and detecting a target nucleic acid. The kit can comprise (i) a CRISPR/Cas complex comprising an effector nuclease and a guide RNA encoding a nucleic acid that hybridizes to a target nucleic acid, (ii) one or more nucleases, (ii) one or more oligonucleotides, and (iv) a fluorescence reporter. Preferably, the one or more nucleases is different to the effector nuclease.

Generally, a CRISPR/Cas complex as used herein comprises an effector nuclease and a guide RNA. The effector nuclease may include a Cas protein from a CRISPR/Cas complex. The Cas protein can be a Cas12 protein or a Cas13 protein. A preferable Cas12 protein is Cas12p. The Cas12p protein can comprise an amino acid sequence that has at least 70% identity to SEQ ID NO: 6. The guide RNA is designed to detect a single nucleotide polymorphism in a target nucleic acid or a splice variant of an RNA transcript.

The method disclosed herein includes providing to a sample one or more nucleases. The one or more nucleases is typically an unspecific nuclease. Exemplary nucleases that are suitable for the methods disclosed herein include Csx1, Cap4, Can1, NucC, or combinations thereof. Without being bound by theory, it is believed that the one or more nucleases is activated by one or more oligonucleotides that is provided to the sample. The oligonucleotides are generally cleaved when the effector nuclease cleaves the target nucleic acid. Cleavage of the target nucleic acid generates the cleavage of one or more oligonucleotides by the collateral activity that act as a second messenger and then activates one or more nucleases. The one or more oligonucleotides can be a cyclic oligonucleotide, a linear oligonucleotide, a polynucleotide, or combinations thereof. The oligonucleotide can be a synthetic oligonucleotide.

The guide RNA in the CRISPR/Cas complex is directed to and can cleave the target nucleic acid. The target nucleic acid can be any RNA or DNA molecule. The target nucleic acid may be a single stranded RNA or a double stranded RNA. The target nucleic acid may be a single stranded DNA or a double stranded DNA.

The target nucleic acid can be from any source. The target nucleic acid can be a viral nucleic acid. The target nucleic acid can be a bacterial nucleic acid. The target nucleic acid can be a fungal nucleic acid. The target nucleic acid can be from a parasite. The target nucleic acid can be from a protozoa. While the target nucleic acid can be from any source, viral nucleic acids are particularly suitable for the methods disclosed herein.

Viral nucleic acids can be from a DNA virus, an RNA virus, or a retrovirus. The viral nucleic acid can be from a Myoviridae, a Podoviridae, a Siphoviridae, an Alloherpesviridae, a Herpesviridae, a Malocoherpesviridae, a Lipothrixviridae, a Rudiviridae, an Adenoviridae, an Ampullaviridae, an Ascoviridae, an Asfarviridae, a Baculoviridae, a Cicaudaviridae, a Clavaviridae, a Corticoviridae, a Fuselloviridae, a Globuloviridae, a Guttaviridae, a Hytrosaviridae, a Iridoviridae, a Maseilleviridae, a Mimiviridae, a Nudiviridae, a Nimaviridae, a Pandoraviridae, a Papillomaviridae, a Phycodnaviridae, a Plasmaviridae, a Polydnaviruses, a Polyomaviridae, a Poxviridae, a Sphaerolipoviridae, a Tectiviridae, a Turriviridae, a Dinodnavirus, a Salterprovirus, a Rhizidovirus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, a Deltavirus, or combinations thereof. A preferable viral nucleic acid can be from severe acute respiratory syndrome coronavirus 2 (SARS-CoV-2) or influenza.

The bacterial nucleic acid can be from an Acinetobacter, an Actinobacillus, an Actinomycete, an Actinomyces, an Aerococcus, an Aeromonas, an Anaplasma, an Alcaligenes, a Bacillus, a Bacteroides, a Bartonella, a Bifidobacterium, a Bordetella, a Borrelia, a Brucella, a Burkholderia, a Campylobacter, a Capnocytophaga, a Chlamydia, a Citrobacter, a Coxiella, a Corynbacterium, a Clostridium, an Eikenella, an Enterobacter, an Escherichia, an Enterococcus, an Ehlichia, an Epidermophyton, an Erysipelothrix, a Eubacterium, a Francisella, a Fusobacterium, a Gardnerella, a Gemella, a Haemophilus, a Helicobacter, a Kingella, a Klebsiella, a Lactobacillus, a Lactococcus, a Listeria, a Leptospira, a Legionella, a Leptospira, Leuconostoc, a Mannheimia, a Microsporum, a Micrococcus, a Moraxella, a Morganell, a Mobiluncus, a Micrococcus, Mycobacterium, a Mycoplasm, a Nocardia, a Neisseria, a Pasteurelaa, a Pediococcus, a Peptostreptococcus, a Pityrosporum, a Plesiomonas, a Prevotella, a Porphyromonas, a Proteus, a Providencia, a Pseudomonas, a Propionibacteriums, a Rhodococcus, a Rickettsia, a Rhodococcus, a Serratia, a Stenotrophomonas, a Salmonella, a Serratia, a Shigella, a Staphylococcus, a Streptococcus, a Spirillum, a Streptobacillus, a Treponema, a Tropheryma, a Trichophyton, a Ureaplasma, a Veillonella, a Vibrio, a Yersinia, a Xanthomonas, or combinations thereof.

The fungal nucleic acid can be from Aspergillus, Blastomyces, Candidiasis, Coccidiodomycosis, Cryptococcus neqformans, Cryptococcus gatti, sp. Histoplasma, Pneumocystis sp., Stachybotrys, Mucroymcosis, Sporothrix, Exserohilum, Cladosporium, Geotrichum, Saccharomyces, Hansenula, Candida, Kluyveromyces, Debaryomyces, Pichia, Penicillium, Cladosporium, Byssochlamys or a combination thereof.

The parasitic nucleic acid can be from Trypanosoma cruzi, T. brucei gambiense, T. brucei rhodesiense, Leishmania braziliensis, L. infantum, L. mexicana, L. major, L. tropica, L. donovani, Naegleria fowleri, Giardia intestinalis (G. lamblia, G. duodenalis), canthamoeba castellanii, Balamuthia madrillaris, Entamoeba histolytica, Blastocystic hominis, Babesia microti, Cryptosporidium parvum, Cyclospora cayetanensis, Plasmodium falciparum, P. vivax, P. ovale, P. malariae, and Toxoplasma gondii, or combinations thereof.

The protozoan nucleic acid can be from a Euglenozoa, a Heterolobosea, a Diplomonadida, an Amoebozoa, a Blastocystic, an Apicomplexa, or combinations thereof.

The methods disclosed herein can also be suitable for detecting a disease. The disease may be an autoimmune disease, cancer, or an infection. The infection may be caused by a virus, a bacterium, a fungus, a protozoa, or a parasite. The viral infection can be caused by Coronavirus, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza virus, or Hepatitis D virus.

The methods disclosed herein can exhibit an attomolar (aM) sensitivity detection. The methods disclosed herein can detect a target nucleic acid in a sample at a concentration of 2 aM or greater.

The fluorescence reporter is used as an indicator to detect the presence of a fluorescence signal. The fluorescence reporter can be a FAM-Q reporter. For fluorescent detection, detection can be performed either as an endpoint readout or in real time using a fluorescent optical detection system including but not limited to fluorometers, spectrophotometers, microplate readers, photodetectors, and light dependent resistors.

The methods disclosed herein can be carried out in vitro, ex vivo, or in vivo. The method disclosed herein can be carried out at a single temperature. Alternatively, the method disclosed herein can be carried out at different temperatures.

The sample can be blood, plasma, serum, saliva, urine, stool, sputum, mucous, a tissue biopsy, lymph fluid, synovial fluid, bile, ascites, pleural effusion, seroma, saliva, cerebrospinal fluid, aqueous or vitreous humor, or any bodily secretion, a transudate, an exudate, fluid obtained from a joint, or a swab of skin or mucosal membrane surface.

### BRIEF DESCRIPTION OF DRAWINGS

FIG. 1 is a general schematic of the CRISPR/Cas work-flow for identifying a target nucleic acid. The assay uses a CRISPR/Cas complex that has collateral activity when activated by cleavage of the target nucleic acid. The collateral activity generates a second messenger which activates a nuclease. The nuclease amplifies the fluorescence in the presence of a target nucleic acid.

FIG. 2 is a general scheme using a Cas13 effector nuclease and a Csx1 for amplification of the fluorescence signal.

FIG. 3 shows a gel from an electrophoresis of affinity purified Csx1.

FIG. 4 shows a graph and a gel of SisCsx-1 from a size exclusion chromatograph.

FIG. 5 is a graph showing a derivative melting curve of Csx1 from a thermal shift assay.

FIGS. 6A-6B are graphs showing Csx1 amplification activity complemented Cas13 at 37° C. FIGS. 6A-6B show the fluorescence for Csx1 alone (the nuclease), LwaCas13 alone (the CRISPR/Cas complex), and Csx1 with LwaCas13a at 30 minutes (FIG. 6A) and at 60 minutes (FIG. 6B). A 100% signal increase was observed when the nuclease and the CRISPR/Cas complex were combined.

FIGS. 7A-7B are graphs showing Csx1 amplification activity complemented with Cas13 at 42° C. FIGS. 7A-78 show the fluorescence for Csx1 alone (the nuclease), LwaCas13 alone (the CRISPR/Cas complex), and Csx1 with LwaCas13a at 30 minutes. A 300% signal increase was observed when the nuclease and the CRISPR/Cas complex were combined.

FIGS. 8A-8B are mass spectrometry readings showing that the Cas13 activated a variety of second messengers with its collateral activity. FIG. 8A discloses SEQ ID NOs 9-14, respectively, in order of appearance.

FIG. 9 is a general scheme using a Cas12 effector nuclease and a Csx1 for amplification of the fluorescence signal.

FIGS. 10A-10B are graphs showing Csx1 amplification activity complemented Cas12 at 37° C. FIGS. 10A-10B show the fluorescence for Csx1 alone (the nuclease), Cas12p alone (the CRISPR/Cas complex), and Csx1 with Cas12p at 30 minutes (FIG. 10A) and at 60 minutes (FIG. 10B). A 100% signal increase was observed when the nuclease and the CRISPR/Cas complex were combined.

FIGS. 11A-11B are mass spectrometry readings showing that the Cas12p activated a variety of second messengers with its collateral activity. FIG. 11A discloses SEQ ID NOs 9-14, respectively, in order of appearance.

FIGS. 12A-12B shows NucC cleavage activity for double-stranded DNA (FIG. 12A) and single-stranded DNA (FIG. 12B) on a gel.

FIG. 13 shows a gel that demonstrates that NucC enzyme was activated with its cyclic RNA activator (c-triAMP).

FIG. 14A shows time courses of activation of SyCsx1 nuclease by cyclic tetraAMP (cA4) and linear tetraAMP>P (rA4>P). FIG. 14B shows time courses of activation of SyCsx1 nuclease by cyclic triAMP (cA3), cA4, cyclic hexaAMP (cA6), linear triAMP>P (rA3>P), and rA4>P.

FIG. 15 shows time courses of activation of PfuCsx1 nuclease by cA4 and rA4>P.

FIG. 16 shows time courses of activation of TtCsm6 nuclease by cA4 and rA4>P.

FIG. 17 shows time courses of activation of SyCsx1 nuclease by various concentrations of rA4>P.

FIG. 18 shows time courses of activation of PfuCsx1 nuclease by various concentrations of rA4>P.

FIG. 19 shows time courses of activation of TtCsm6 nuclease by various concentrations of rA4>P.

FIG. 20 shows time courses of activation of SyCsx1 nuclease by cA4 as indicated by various FAM-Q reporters.

FIG. 21 shows time courses of activation of PfuCsx1 nuclease by cA4 as indicated by various FAM-Q reporters.

FIG. 22 shows time courses of activation of PfuCsx1 nuclease by cA4 as indicated by various FAM-Q reporters.

FIG. 23 shows time courses of detection of isolated SARS RNA by LbuCas13a and SyCsx1 with 2 μM rA4(rU5) and FAM-Q polyC ssRNA reporter.

FIG. 24 shows time courses of detection of isolated SARS RNA by LbuCas13a and SyCsx1 with 5 μM rA4(rU5) and 1 μM rA4(1-2*)(rC5), and FAM-Q polyC ssRNA reporter.

FIG. 25 shows time courses of detection of isolated SARS RNA by LbuCas13a and TtCsm6 with 2 μM rA4(rU5) and FAM-Q UCU ssRNA reporter.

FIG. 26 is a general schematic of a CRISPR/Cas work-flow for identifying a target nucleic acid, making use of a cyclic pre-second messenger.

### DETAILED DESCRIPTION

The disclosure relates to novel methods for rapidly identifying a target nucleic acid in a sample using a CRISPR/Cas system that exhibits collateral activity. The methods disclosed herein have high-specificity, sensitivity, and accuracy. In addition, the methods can be performed in a single system without needing to perform a pre-amplification step, as is required by other known methods.

The methods disclosed herein comprise providing to a sample a CRISPR/Cas complex that comprises an effector nuclease and a guide RNA. The CRISPR/Cas complex exhibits target-dependent promiscuous cleavage activity and collateral activity. The effector nuclease typically includes a Cas protein, for example Cas12 or Cas13. Cas12p is a preferable Cas12 protein that is suitable for the methods disclosed herein. The Cas protein generally comprises at least one domain that interacts with the guide RNA. Additionally, the Cas protein is typically directed to a target nucleic acid sequence by the guide RNA. The guide RNA interacts with the Cas protein as well as the target nucleic acid sequence such that, once directed to the target sequence, the Cas protein is capable of cleaving the target nucleic acid sequence. The Cas protein can be either a RNA or DNA effector nuclease. The guide RNA provides the specificity for the targeted cleavage of the target nucleic acid. The Cas protein may be paired with different guide RNAs to cleave different target sequences.

The method may further comprise providing to the sample one or more nucleases, one or more oligonucleotides, and a fluorescence reporter. Preferably, the one or more nucleases is not the same as the effector nuclease.

Once the CRISPR/Cas complex has been activated by cleavage of a target nucleic acid, the CRISPR/Cas complex becomes a nuclease and promiscuously cleaves the one or more oligonucleotides through its collateral activity. The result is that the oligonucleotides in the sample can be cleaved. Once the oligonucleotides have been cleaved by the collateral activity of the CRISPR/Cas complex, the oligonucleotides act as a second messenger and activate one or more nucleases. The one or more nucleases is preferably an unspecific nuclease. The one or more nucleases can cleave the fluorescence reporter, which amplifies the fluorescence signal. When a target nucleic acid is present the fluorescence signal can be detected.

The methods disclosed herein can be used to detect any suitable target nucleic acid. For example a viral nucleic acid, a bacterial nucleic acid, a parasitic nucleic acid, a fungal nucleic acid, or a protozoan nucleic acid. The methods disclosed herein can also be used to detect a disease and/or identify a subject having a target nucleic acid.

In embodiments, the methods disclosed herein do not use a Cas13 protein in combination with a Csm6 nuclease.

Further disclosure relating to the methods provided herein is provided below.

### A. Definitions

Certain illustrative and preferred embodiments are described in detail herein. The embodiments within the specification should not be construed to limit the scope of the disclosure.

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “hybridize” refers to a nucleic acid (e.g., a DNA or an RNA) that comprises a sequence of nucleotides that enables it to non-covalently bind to another nucleic acid sequence in a sequence-specific, antiparallel manner under the appropriate conditions.

The term “nucleic acid” refers to a polymeric form of nucleotides of any length, either ribonucleotides or deoxyribonucleotides. Nucleic acid encompasses single-stranded DNA, double-stranded DNA, multi-stranded DNA, single-stranded RNA, double-stranded RNA, multi-stranded RNA, genomic DNA, cDNA, DNA-RNA hybrids, and a polymer comprising purine and pyrimidine bases or other natural, chemically or biochemically modified, non-natural, or derivatized nucleotide bases.

The term “subject” as used herein refers to any animal, such as any mammal, including but not limited to, humans, non-human primates, rodents, and the like. In some embodiments, the mammal is a mouse. In some embodiments, the mammal is a human.

It is appreciated that certain features of the invention, which are, for clarity, described in the context of separate embodiments, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every sub-combination was individually and explicitly disclosed herein.

### B. Overview

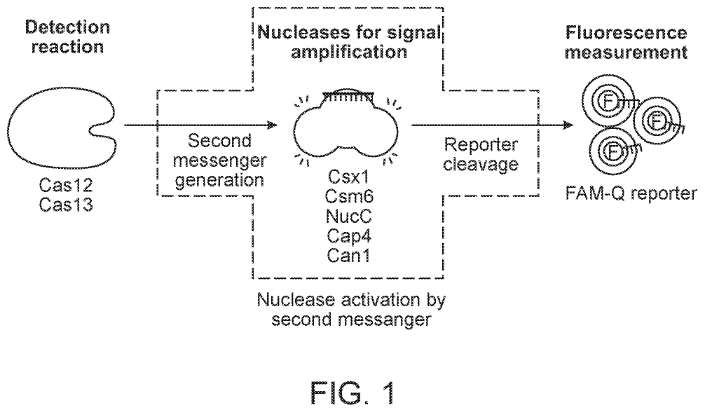

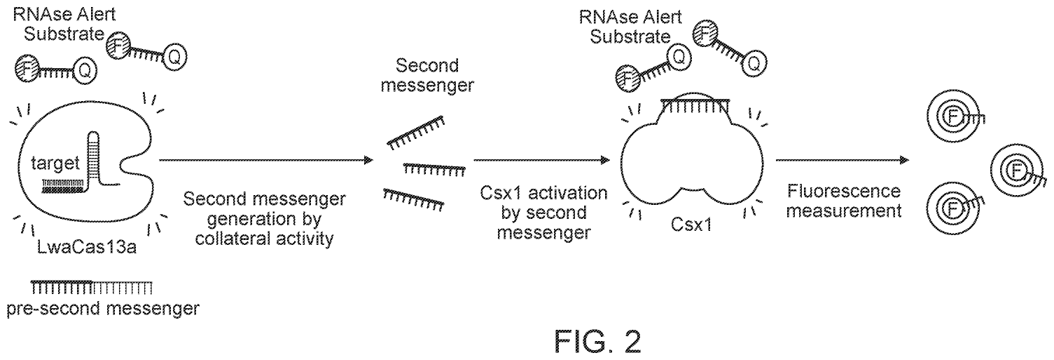

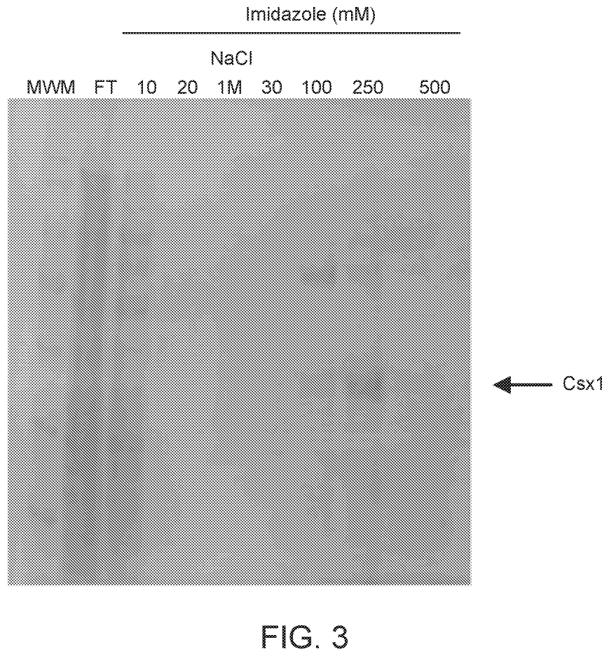

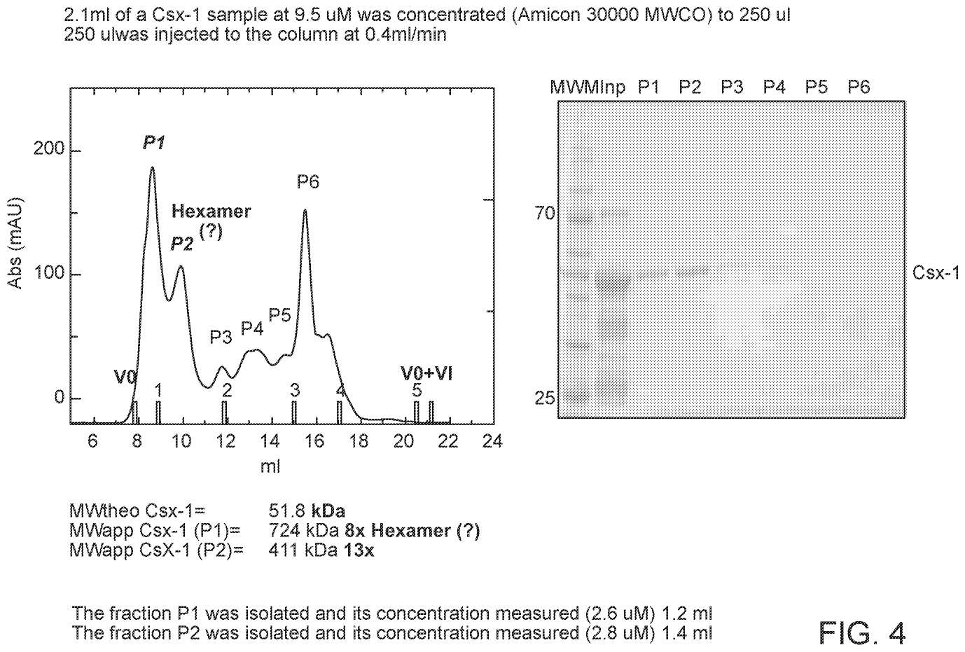

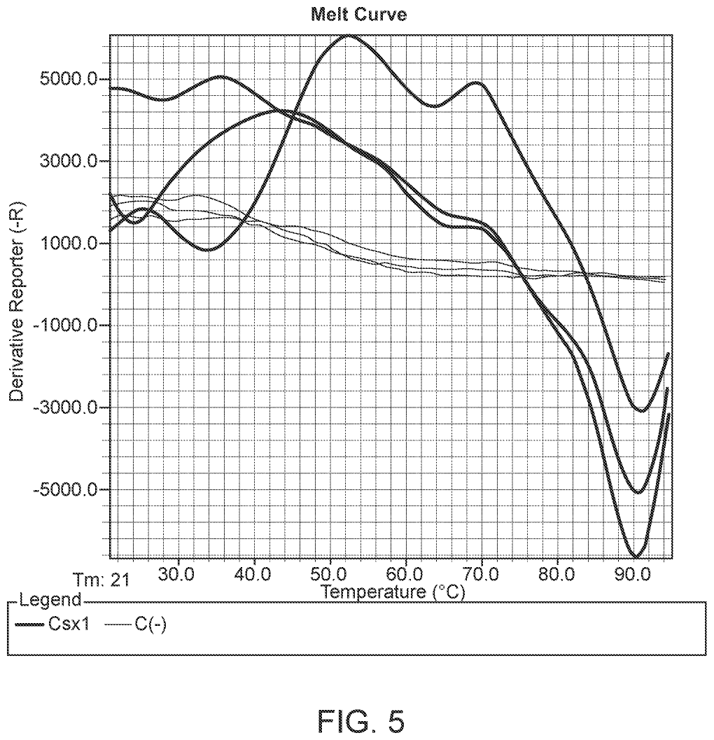

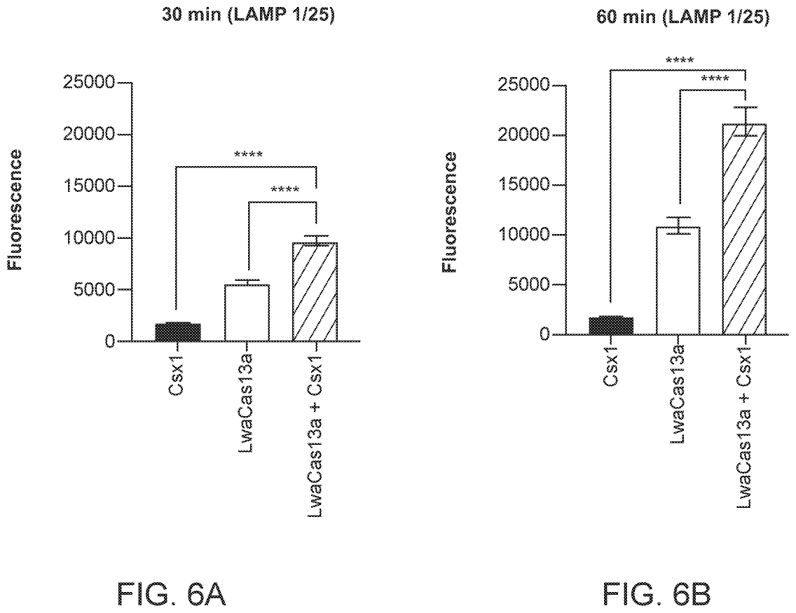

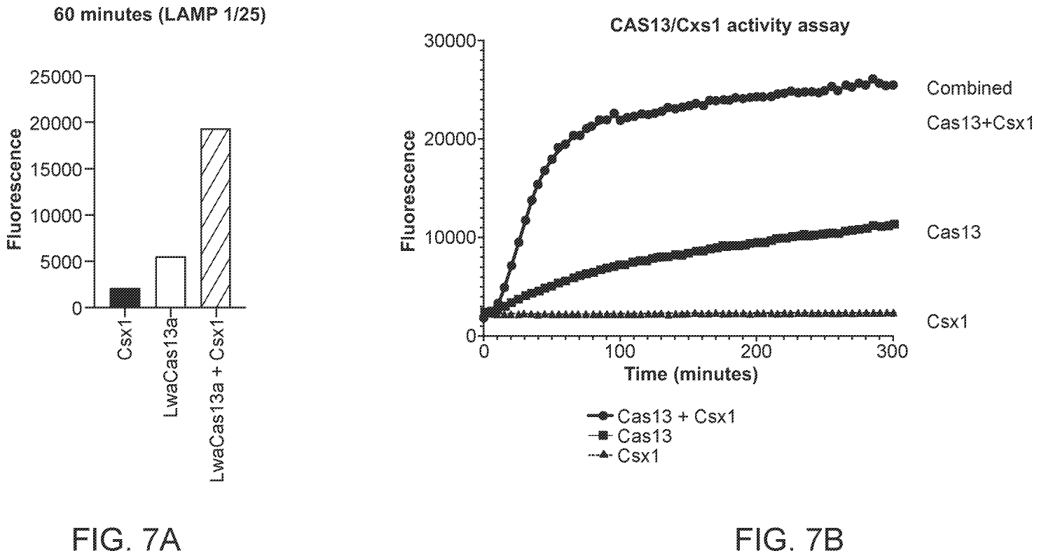

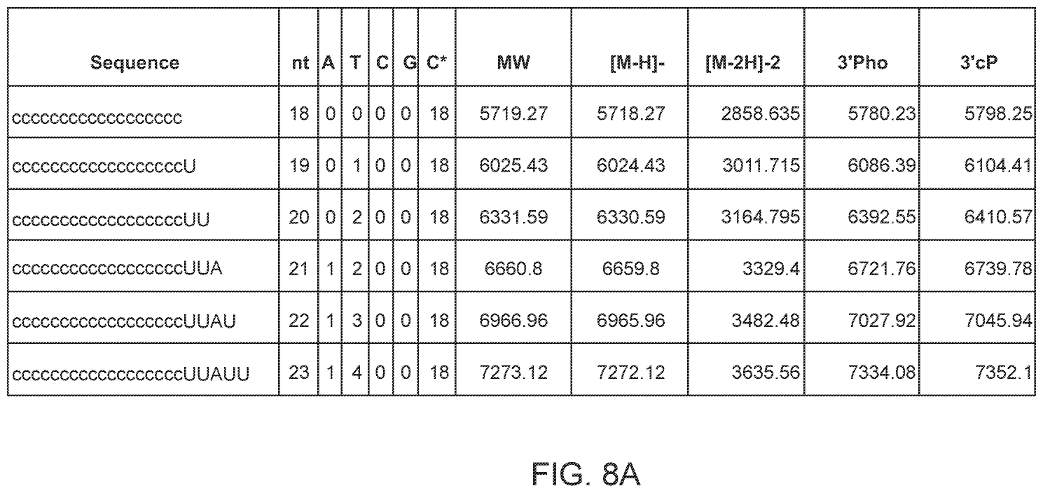

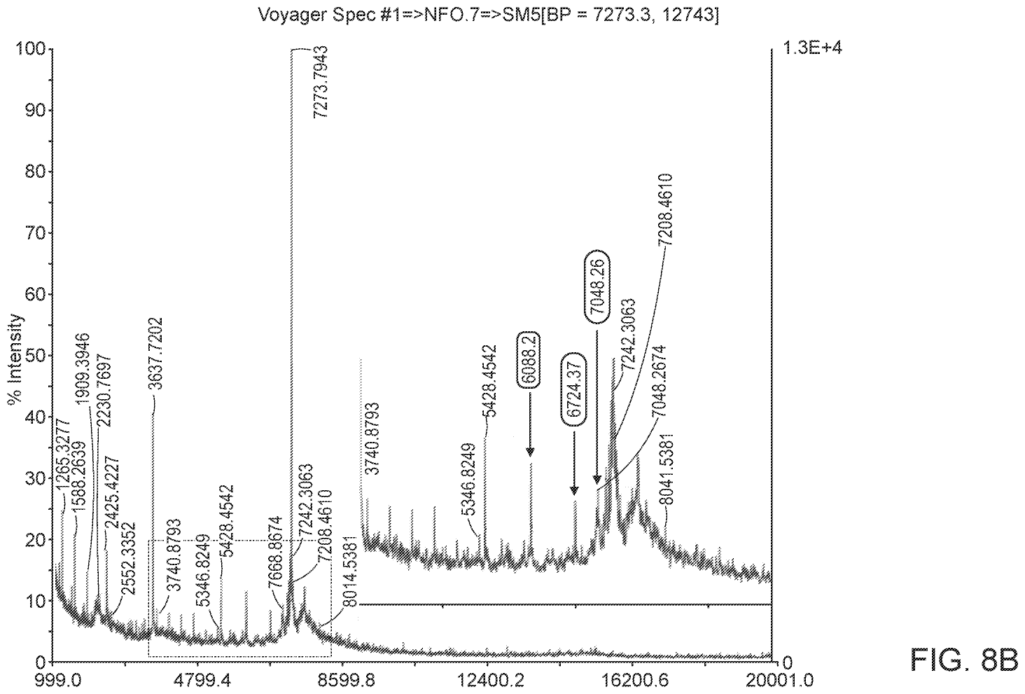

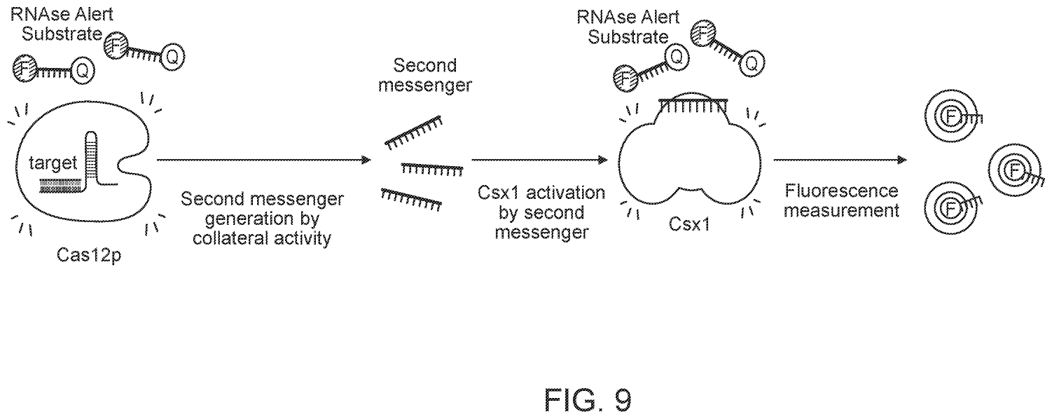

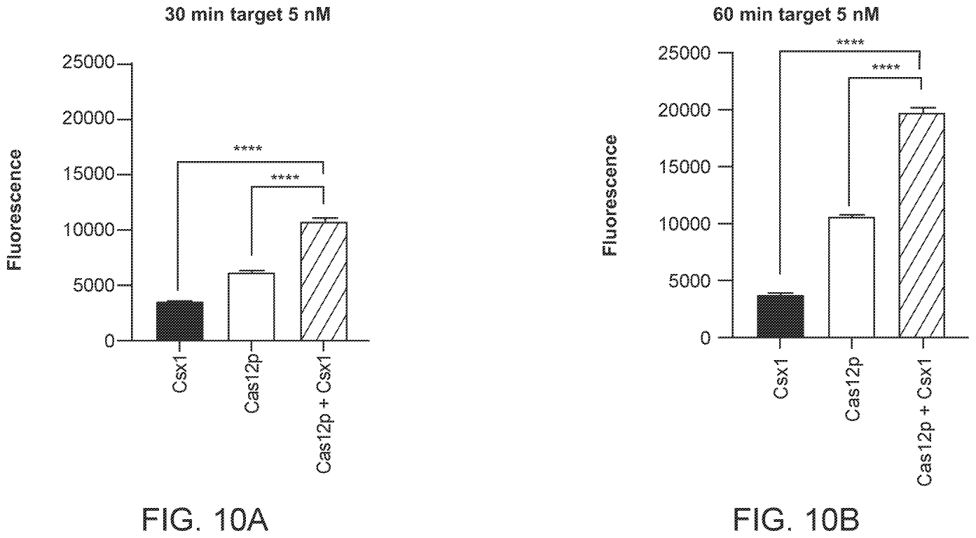

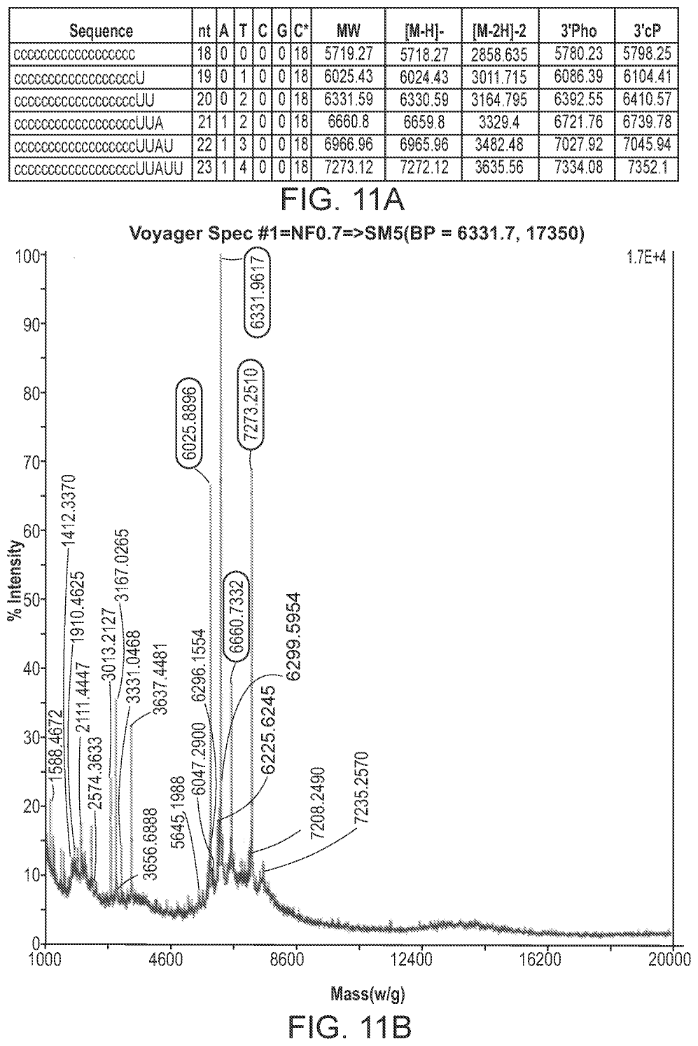

FIG. 1, FIG. 2, FIG. 9, and FIG. 26 show general schema of the methods for detecting a target nucleic acid disclosed herein. Generally, a Cas nuclease, such as a Cas12 or a Cas13 (FIG. 1 and FIG. 26), for example, LwaCas13a (FIG. 2) or a Cas12p (FIG. 9), in a complex with a guide RNA, binds to a target DNA or RNA molecule (e.g., FIG. 26, point 1). The Cas nuclease is activated, cleaving the target molecule, and non-specifically cleaving bystander oligonucleotides, such as the pre-second messenger shown in FIG. 2 and/or the balloon shown in FIG. 26, thereby yielding the second messenger shown in FIG. 2 and/or the physiological activator shown in FIG. 26.

The second messenger/physiological activator activates a nuclease other than the Cas nuclease. Examples of such nucleases include Csx1, NucC, Cap4, and Can1 (FIG. 1). Upon activation, the nuclease cleaves a reporter molecule, such as a FAM-Q reporter (FIG. 1) or other reporter molecule which generates a signal after cleavage. The generated signal is thus indicative of the presence of the target molecule.

This general schema and its various components are described below in more detail.

### C. CRISPR/Cas Complex

i. CRISPR/Cas

As described above, the inventors have developed methods for detecting a target nucleic acid in a single system using a CRISPR/Cas complex. Generally, a CRISPR/Cas complex as used herein comprises an effector nuclease and a guide RNA.

The effector nuclease may include a Cas protein (also called a “Cas nuclease”) from a CRISPR/Cas complex. The Cas protein may comprise at least one domain that interacts with a guide RNA. Additionally, the Cas protein is typically directed to a target nucleic acid sequence by the guide RNA. The guide RNA interacts with the Cas protein as well as the target nucleic acid sequence such that, once directed to the target sequence, the Cas protein is capable of cleaving the target nucleic acid sequence. The Cas protein can be either a RNA or DNA effector nuclease. The guide RNA provides the specificity for the targeted cleavage of the target nucleic acid. The Cas protein may be paired with different guide RNAs to cleave different target sequences. The effector nuclease and the target nucleic acid typically do not naturally occur together.

The CRISPR/Cas system may be a Class 1 having Types I, III, and IV or Class 2 having types II, V, VI. See, e.g., Mararova et al., Nat Rev Microbiol, 13(11): 722-36 (2015). The CRISPR-Cas system may be an RNA guided endonuclease.

The CRISPR-Cas system suitable for the method described herein exhibits target-dependent promiscuous RNase/DNase activity, leading to trans cleavage of bystander RNA molecules, an effect termed “collateral activity.” See, e.g., Abudayyeh et al. Science 353(6299) (2016), Li et al., Cell Res 28, 491-493, (2018); and Chen et al., Science, 360(6387):436-439, (2018). Type V and Type VI effector nucleases exhibit collateral activity. Id.

The effector nuclease suitable for the methods disclosed herein may be a Cas12 protein, a Cas13 protein, or variants thereof. Cas12 can encompass Cas12a, Cas12b, Cas12c, Cas12d, Cas12e, Cas12f, Cas12g, Cas12h, Cas12i, Cas12j, Cas12k, Cas12p, or variants thereof. Cas13 can encompass Cas13a, Cas13b, Cas13c, Cas13d, or variants thereof. Preferably, the effector nuclease is Cas12p or a variant thereof. The Cas12p protein or variant thereof can comprise an amino acid sequence that has at least about 70% identity to SEQ ID NO: 6. For example, the Cas12p protein or variant thereof can comprise an amino acid sequence that has at least 70%, at least 75%, at least 80%, at least 85%, at least 90%, at least 95%, or greater identity to SEQ ID NO: 6. The Cas12p protein or variant thereof can comprise a nucleic acid sequence that has at least about 70% identity to SEQ ID NO: 7. For example, the Cas12p protein or variant thereof can comprise an amino acid sequence that has at least 70%, at least 75%, at least 80%, at least 85%, at least 90%, at least 95%, or greater identity to SEQ ID NO: 7. The variants of Cas12 or Cas13 can share certain structural, sequence, or functional similarities with any one of the subtypes of Cas12 or Cas13. Type V effector nucleases, such as Cas12, are capable of cleaving target single stranded DNA or double stranded DNA.

Cas13 specifically recognizes and cleaves only RNA. Cas13 exhibits target-dependent promiscuous RNase activity, leading to trans cleavage of bystander RNA molecules. Many of the Cas13 subtypes and orthologs have different preferences, cleaving at specific dinucleotide motifs. In addition, Cas13 subtypes differ in size, direct repeat sequence, and CRISPR RNA structure. Although Cas13 has a protospacer adjacent motif (PAM)-like sequence called the protospacer flanking site (PFS) that restricts activity to only certain target sites, there are a number of Cas13 orthologs, such as LwaCas13a, that show no PFS. Lack of a protospacer flanking site (note that for RNA-targeting and RNA-cleaving Cas effectors, the PFS, instead of the PAM sequence, is typically necessary for target RNA binding and cleaving) is a distinguishing feature of these orthologs that enables them to target any possible sequence or mutation.

In embodiments, the methods disclosed herein do not use a Cas13 protein in combination with a Csm6 nuclease.

Cas12 typically recognizes and cleaves a specific DNA target. A Cas12 effector nuclease may comprise one or more RuvC motifs, which is thought to be responsible for its catalytic activity. For example the Cas12 may comprise about 1, about 2, about 3, about 4, or about 5 RuvC motifs.

Non-limiting species that the Cas protein or other components of the CRISPR/Cas complex may be from include Streptococcus pyogenes, Streptococcus thermophilics, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polar omonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodular ia spumigena, Nostoc sp., Arthrospira maxima, Arthrospira platensis, Arthrospira sp., Lyngbya sp., Microcoleus chthonoplastes, Oscillator ia sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina.

ii. Guide RNA

As described herein, the CRISPR/Cas complex comprises a guide RNA. The guide RNA guides the effector nuclease (e.g., the Cas protein) to a target nucleic acid. The guide RNA and the effector nuclease (e.g., the Cas protein) may form a ribonucleoprotein (RNP), for example a CRISPR/Cas complex. The guide RNA hybridizes with and the effector nuclease (e.g., the Cas protein) cleaves the target sequence.

The guide RNA for a CRISPR/Cas complex may comprise a CRISPR RNA (crRNA) and/or a tracr RNA. The crRNA comprises a nucleic acid sequence that recognizes and hybridizes to a target nucleic acid. The tracr RNA typically serves as a binding scaffold for the Cas nuclease.

In embodiments, the crRNA may comprise a targeting sequence that is complementary to and hybridizes with the target nucleic acid. The crRNA may also comprise a flagpole that is complementary to and hybridizes with a portion of the tracr RNA. The crRNA may parallel the structure of a naturally occurring crRNA transcribed from a CRISPR locus of a bacteria, whereas the targeting sequence acts as the spacer of the CRISPR/Cas system, and the flagpole corresponds to a portion of a repeat sequence flanking the spacers on the CRISPR locus.

The guide RNA may target any sequence of interest. The degree of complementarity between the guide RNA and the target nucleic acid can be about 50%, about 55%, about 60%, about 65%, about 70%, about 75%, about 80%, about 85%, about 90%, about 95%, about 96%, about 97%, about 98%, about 99%, or about 100%. For example, the guide RNA and the target nucleic acid may be 100% complimentary. The guide RNA and the target nucleic acid sequence are typically at least about 90% or greater complimentary. The guide RNA and the target nucleic acid sequence may contain at least one mismatch. For example, the target nucleic acid sequence and the guide RNA may contain 1, 2, 3, 4, 5, 6, 7, 8, 9, or 10 mismatches.

The length of the guide RNA may depend on the CRISPR/Cas complex used or the length of the target nucleic acid. For example, the guide RNA may comprise about 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70 or more than 70 nucleotides. For example, the guide RNA may comprise about 18-24 nucleotides. For example, the guide RNA may comprise about 20-35 nucleotides. For example, the guide RNA may comprise about 40-70 nucleotides. For example, the guide RNA may comprise about 100-150 nucleotides.

In general, the guide RNA may comprise a single RNA molecule (“single guide RNA”). Alternatively, the guide RNA may optionally comprise two RNA guides (“dual guide RNA”). A dual guide RNA may comprise a first RNA molecule comprising a cRNA and a second RNA molecule comprising a tracr RNA. The first and second RNA molecules may form a RNA duplex via the base pairing between the flagpole on the cRNA and the tracr RNA.

The flagpole may comprise any sequence with sufficient complementarity with a tracr RNA to promote the formation of a functional CRISPR/Cas complex. The flagpole can comprise all or a portion of the sequence of a naturally-occurring crRNA that is complementary to the tracr RNA in the same CRISPR/Cas system. The flagpole may comprise a truncated or modified tag or handle sequence. The degree of complementarity between the tracr RNA and the flagpole that hybridizes with the tracr RNA and the portion of the flagpole that hybridizes with the tracr RNA along the length of the shorter of the two sequences may be about 40%, about 50%, about 60%, about 70%, about 80%, or higher. In embodiments, the tracr RNA and the portion of the flagpole that hybridizes with the tracr RNA are not 100% complementary along the length of the shorter of the two sequences because of the presence of one or more bulge structures on the tracr RNA and/or wobble base pairing between the tracr and the flagpole. The length of the flagpole may depend on the CRIPR/Cas complex used or the tracr RNA used. For example, the flagpole may comprise about 10-50 nucleotides or more than 50 nucleotides.

The tracr RNA may comprise all or a portion of a wild-type tracr RNA. The length of the tracr RNA may depend on the CRISPR/Cas complex used. The tracr RNA may comprise about 5, 6, 7, 8, 9, 10, 11, 12, 13, 14, 15, 16, 17, 18, 19, 20, 25, 30, 40, 50, 60, 70, 80, 90, 100, or more nucleotides. For example, the tracr RNA may be at least 40 nucleotides in length. For example, the tracr RNA may be at least 60 nucleotides in length. For example, the tracr RNA may be at least 80 nucleotides in length.

### D. Second Messengers

The methods disclosed herein comprise providing to a sample one or more oligonucleotides. Without wishing to be bound by theory or mechanism, it is believed that once the CRISPR/Cas complex is activated by the guide RNA, which occurs when a sample includes the target nucleic sequence and the guide RNA hybridizes, the CRISPR/Cas complex cleaves one or more oligonucleotides not comprising the target nucleic acid. The oligonucleotides act as a second messenger and activate one or more nucleases.

The one or more oligonucleotides can be a cyclic oligonucleotide, a linear oligonucleotide, a polynucleotide, or combinations thereof. The oligonucleotide can be a synthetic oligonucleotide.

The oligonucleotide can be RNA or DNA. The oligonucleotide can be a single-stranded DNA, a double-stranded DNA, a single-stranded RNA, a double-stranded RNA, an antisense oligonucleotide, an aptamer RNA, or combinations thereof.

Exemplary cyclic oligonucleotides that are suitable for the methods disclosed herein include cA3, cA4, cA6, cA8, AAG, and AAC.

A cyclic oligonucleotide is an oligonucleotide comprising, but not necessarily consisting of, a cyclic moiety. For example, the molecule labeled “balloon” in FIG. 26 is a cyclic oligonucleotide comprising cyclic triAMP (cA3) and a linear tail. Upon cleavage of the linear tail from the cA3 by a Cas enzyme, the cA3 activates the nuclease.

The oligonucleotide may be a 2-mer, a 3-mer, a 4-mer, a 5-mer, a 6-mer, a 7-mer, a 8-mer, a 9-mer, a 10-mer, a 11-mer, a 12-mer, a 13-mer, a 14-mer, a 15-mer, a 16-mer, a 17-mer, a 18-mer, a 19-mer, a 20-mer, a 21-mer, a 22-mer, a 23-mer, a 24-mer, a 25-mer, a 26-mer, a 27-mer, a 28-mer, a 29-mer, a 30-mer, a 31-mer, a 32-mer, a 33-mer, a 34-mer, a 35-mer, a 36-mer, a 37-mer, a 38-mer, a 39-mer, a 40-mer, a 41-mer, a 42-mer, a 43-mer, a 44-mer, a 45-mer, a 46-mer, a 47-mer, a 48-mer, a 49-mer, a 50-mer, or longer. Typically, the oligonucleotide is an 2-mer to about a 50-mer.

The oligonucleotide may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 or longer nucleotides in length. Typically, the oligonucleotide is about 2 to about 50 nucleotides in length.

### E. Nucleases

The methods disclosed herein comprise one or more nucleases. Without being bound by theory or mechanism, it is believed that the one or more oligonucleotides act as second messengers and activate the one or more nucleases. The one or more nucleases cleaves the fluorescence reporter.

Nucleases used in the methods disclosed herein may be an exonuclease or an endonuclease. Endonucleases are particularly suitable for the methods disclosed herein. Exonucleases are capable of cleaving nucleotides one at a time from the end of a polynucleotide chain.

The endonuclease can be a deoxyribonuclease or a ribonuclease. Preferably, the nucleases used in the methods disclosed herein are unspecific nucleases. The nuclease may cleave a single stranded RNA, double stranded RNA, a single stranded DNA, a double stranded DNA, or combinations thereof.

Exemplary, non-limiting, nucleases that can be suitable for the methods disclosed herein include Csx1, Cap4, Can, NucC, Csm6, Eco RI, EcoRV, BamHI, PvuII, RuvC, Bal31, Dnase I, HindII, SI nuclease, ligase, micrococcal nuclease, Flap endonuclease 1, Mre11, Trex1, Trex2, ExoI, ExoxX, RNase T, RNase Orn, RNase D, RNase Rrp6, PARN, Pan2, Pop2, ERI-1, 3′hExo, CRN-4, RNase H1, RNAase H2, argonaute, Ydc2, UvrC, HincII, MutH, EcoR124, Phage 2, RecE, RecB, AdnAb, HJ resolvase, XPF, Hef, Rad1, Mus81, Vsr, Rail/Dom3Z, FEN1, Exo1, XRN2, XRN1, PIN domain RNA exonuclease, RecJ, DHH, TOPRIM nuclease, DNAase I, AP endonuclease Exo III, APEI, RNase E, RNAase G, beta-CASP, metallo-beta-lactamase, Rnase Z, CPSF-73, RNase J1, Artemis, Snm1, Pos2, Mre11, protein phosphatase 2B fold, LAGLIDADG homing endonuclease (“LAGLIDADG” disclosed as SEQ ID NO: 8), Rnase II, Rrp44, RNase III, Dicer, RNase PH, PNPase, exosome, RusA, or combinations thereof.

Cap4 (for example, AbCap4 or EcCap4), Csx1, Can1, and NucC are preferable nucleases that can be used in the methods disclosed herein. The Csx1 nuclease can comprise an amino acid sequence that has at least 70% identity to SEQ ID NO: 1. For example, The Csx1 nuclease can comprise an amino acid sequence that has at least 70%, at least 75%, at least 80%, at least 85%, at least 90%, at least 95%, or greater identity to SEQ ID NO: 1. The NucC nuclease can comprise an amino acid sequence that has at least 70% identity to SEQ ID NO: 2. For example, the NucC nuclease can comprise an amino acid sequence that has at least 70%, at least 75%, at least 80%, at least 85%, at least 90%, at least 95%, or greater identity to SEQ ID NO: 2. The Can1 nuclease can comprise an amino acid sequence that has at least 70% identity to SEQ ID NO: 3. For example, the Can1 nuclease can comprise an amino acid sequence that has at least 70%, at least 75%, at least 80%, at least 85%, at least 90%, at least 95%, or greater identity to SEQ ID NO: 3. The AbCap4 nuclease can comprise an amino acid sequence that has at least 70% identity to SEQ ID NO: 4. For example, the AbCap4 nuclease can comprise an amino acid sequence that has at least 70%, at least 75%, at least 80%, at least 85%, at least 90%, at least 95%, or greater identity to SEQ ID NO: 4. The EcCap4 nuclease can comprise an amino acid sequence that has at least 70% identity to SEQ ID NO: 5. For example, the EcCap4 nuclease can comprise an amino acid sequence that has at least 70%, at least 75%, at least 80%, at least 85%, at least 90%, at least 95%, or greater identity to SEQ ID NO: 5.

In embodiments, the methods disclosed herein do not use a Cas13 protein in combination with a Csm6 nuclease.

The nuclease may be optionally modified from its wild-type counterpart.

### F. Target Nucleic Acid

The methods disclosed herein comprise detecting the presence of a target nucleic acid. The guide RNA in the CRISPR/Cas complex is directed to and can cleave the target nucleic acid. For example, the effector nuclease may be directed by the guide RNA to the target nucleic acid sequence, where the guide RNA hybridizes with and the effector nuclease (e.g., a Cas protein) cleaves the target nucleic acid sequence. The target nucleic acid sequence can be complementary to the nucleic acid sequence of the guide RNA. The degree of complementarity between a targeting sequence of a guide RNA and its corresponding target nucleic acid sequence may be about 50%, about 55%, about 60%, about 65%, about 70%, about 75%, about 80%, about 85%, about 90%, about 95%, about 97%, about 98%, about 99%, or about 100%. The target nucleic acid sequence and the guide RNA are typically at least about 90% or greater complimentary. The target nucleic acid sequence and the guide RNA may contain at least one mismatch. For example, the target nucleic acid sequence and the guide RNA may contain 1, 2, 3, 4, 5, 6, 7, 8, 9, or 10 mismatches.

The length of the target nucleic acid sequence may depend on the CRISPR/Cas complex used. For example, the target nucleic acid sequence for a CRISPR/Cas complex may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70 or more that 70 nucleotides. For example, the target nucleic acid sequence may be about 18-24 nucleotides in length. For example, the target nucleic acid sequence may be about 20-35 nucleotides in length. For example, the target nucleic acid sequence may be about 40-70 nucleotides in length. For example, the target nucleic acid sequence may be about 100-150 nucleotides in length.

The target nucleic acid can be any RNA or DNA molecule. The target nucleic acid may be a single stranded RNA or a double stranded RNA. The target nucleic acid may be a single stranded DNA or a double stranded DNA. The target nucleic acid can be exogenous or endogenous to a cell. As used herein, the term “endogenous sequence” refers to a sequence that is native to a cell. The term “exogenous sequence” refers to a sequence that is not native to a cell, or a sequence whose native location in the genome of the cells is in a different location.

The target nucleic acid can be from any source. The target nucleic acid can be a viral nucleic acid. The target nucleic acid can be a bacterial nucleic acid. The target nucleic acid can be a fungal nucleic acid. The target nucleic acid can be from a parasite. The target nucleic acid can be from a protozoa. While the target nucleic acid can be from any source, viral nucleic acids are particularly suitable for the methods disclosed herein. Viral nucleic acids can be from a DNA virus, an RNA virus, or a retrovirus.

The viral nucleic acid can be from a Myoviridae, a Podoviridae, a Siphoviridae, an Alloherpesviridae, a Herpesviridae, a Malocoherpesviridae, a Lipothrixviridae, a Rudiviridae, an Adenoviridae, an Ampullaviridae, an Ascoviridae, an Asfarviridae, a Baculoviridae, a Cicaudaviridae, a Clavaviridae, a Corticoviridae, a Fuselloviridae, a Globuloviridae, a Guttaviridae, a Hytrosaviridae, an Iridoviridae, a Maseilleviridae, a Mimiviridae, a Nudiviridae, a Nimaviridae, a Pandoraviridae, a Papillomaviridae, a Phycodnaviridae, a Plasmaviridae, a Polydnaviruses, a Polyomaviridae, a Poxviridae, a Sphaerolipoviridae, a Tectiviridae, a Turriviridae, a Dinodnavirus, a Salterprovirus, a Rhizidovirus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, a Deltavirus, or combinations thereof.

Exemplary, non-limiting, viral target nucleic acids can be from Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, hepatitis C, hepatitis D. An influenza virus may include, for example, influenza A or influenza B. An HIV may include HIV 1 or HIV 2. The virus may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus, California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatis A/B/C/D/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petitsruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

The target nucleic acid can be a bacterial nucleic acid. Exemplary bacterial nucleic acids can be from a Acinetobacter, a Actinobacillus, a Actinomycete, a Actinomyces, a Aerococcus, a Aeromonas, a Anaplasma, a Alcaligenes, a Bacillus, a Bacteroides, a Bartonella, a Bifidobacterium, a Bordetella, a Borrelia, a Brucella, a Burkholderia, a Campylobacter, a Capnocytophaga, a Chlamydia, a Citrobacter, a Coxiella, a Corynbacterium, a Clostridium, a Eikenella, a Enterobacter, a Escherichia, a Enterococcus, a Ehlichia, a Epidermophyton, a Erysipelothrix, a Eubacterium, a Francisella, a Fusobacterium, a Gardnerella, a Gemella, a Haemophilus, a Helicobacter, a Kingella, a Klebsiella, a Lactobacillus, a Lactococcus, a Listeria, a Leptospira, a Legionella, a Leptospira, Leuconostoc, a Mannheimia, a Microsporum, a Micrococcus, a Moraxella, a Morganell, a Mobiluncus, a Micrococcus, Mycobacterium, a Mycoplasm, a Nocardia, a Neisseria, a Pasteurelaa, a Pediococcus, a Peptostreptococcus, a Pityrosporum, a Plesiomonas, a Prevotella, a Porphyromonas, a Proteus, a Providencia, a Pseudomonas, a Propionibacteriums, a Rhodococcus, a Rickettsia, a Rhodococcus, a Serratia, a Stenotrophomonas, a Salmonella, a Serratia, a Shigella, a Staphylococcus, a Streptococcus, a Spirillum, a Streptobacillus, a Treponema, a Tropheryma, a Trichophyton, a Ureaplasma, a Veillonella, a Vibrio, a Yersinia, a Xanthomonas, or combinations thereof.

The target nucleic acid can be from a fungal nucleic acid. Exemplary, non-limiting, fungal target nucleic acids can be from Aspergillus, Blastomyces, Candidiasis, Coccidiodomycosis, Cryptococcus neqformans, Cryptococcus gatti, sp. Histoplasma, Pneumocystis sp., Stachybotrys, Mucroymcosis, Sporothrix, Exserohilum, Cladosporium, Geotrichum, Saccharomyces, Hansemula, Candida, Kluyveromyces, Debaryomyces, Pichia, Penicillium, Cladosporium, Byssochlamys or a combination thereof.

The target nucleic acid can be from a parasite. Exemplary, non-limiting parasitic nucleic acids can be from Trypanosoma cruzi, T. brucei gambiense, T. brucei rhodesiense, Leishmania braziliensis, L. infantum, L. mexicana, L. major, L. tropica, L. donovani, Naegleria fowleri, Giardia intestinalis (G. lamblia, G. duodenalis), canthamoeba castellanii, Balamuthia madrillaris, Entamoeba histolytica, Blastocystic hominis, Babesia microti, Cryptosporidium parvum, Cyclospora cayetanensis, Plasmodium falciparum, P. vivax, P. ovale, P. malar iae, and Toxoplasma gondii, or combinations thereof.

The target nucleic acid can be from a protozoa. Exemplary, non-limiting protozoan nucleic acids can be from a Euglenozoa, a Heterolobosea, a Diplomonadida, an Amoebozoa, a Blastocystic, an Apicomplexa, or combinations thereof.

### G. Samples

The methods disclosed herein comprise providing to a sample a CRISPR/Cas complex, one or more nucleases, one or more oligonucleotides and a fluorescence reporter. A sample suitable for the methods disclosed herein can include any sample that includes nucleic acids, including DNA or RNA.

The sample suitable for the methods disclosed herein can contain one or more nucleic acids. The sample can include two or more (e.g., 3 or more, 5 or more, 10 or more, 20 or more, 50 or more, 100 or more, 500 or more, 1,000 or more, or 5,000 or more) nucleic acids. The sample can include 5 or more nucleic acids (e.g., 10 or more, 20 or more, 50 or more, 100 or more, 500 or more, 1,000 or more, or 5,000 or more DNA or RNAs) that are the same or differ from one another in sequence. The sample can include 10 or more, 20 or more, 50 or more, 100 or more, 200 or more, 300 or more, 400 or more, 500 or more, 1,000 or more, 2,500 or more, 5,000 or more, 10,000 or more, 50,000 or more, 100,000 or more, 150,000 or more, 200,000 or more, 250,000 or more, 300,000 or more, 350,000 or more, 400,000 or more, 450,000 or more, 500,000 or more, or 100,000,000 or more nucleic acids (e.g., DNA or RNAs).

The sample can include nucleic acids that differ from one another, are the same, or a combination thereof. The sample may include the target nucleic acid or the sample may be devoid of the target nucleic acid.

The sample can be derived from any source. For example, the sample can be a synthetic combination of purified DNA or RNA, a cell lysate, a DNA or RNA-enriched cell lysate, DNA or RNA isolated and/or purified from a cell lysate, or biological material obtained from a subject. Cell lysates can include, but are not limited to, eukaryotic cell lysate, a mammalian cell lysate, a human cell lysate, a prokaryotic cell lysate, or a plant cell lysate.

The sample can be a biological material obtained from human or non-human subjects. Preferentially, the biological material is obtained from a human. Suitable samples include, but are not limited to, blood, plasma, serum, urine, stool, sputum, mucous, lymph fluid, synovial fluid, bile, ascites, pleural effusion, seroma, saliva, cerebrospinal fluid, saliva, aqueous or vitreous humor, any bodily secretion, a transudate, an exudate, fluid obtained from a joint, or a swab of skin or mucosal membrane surface (for example, nasopharyngeal, oropharyngeal, nasopharyngeal/oropharyngeal, aspirate), or a biopsy.

Samples may also be samples that have been manipulated in any way after their procurement, such as by treatment with reagents, washed, or enrichment for certain cell populations, such as cancer cells. The samples can be obtained by use of a swab, for example, a nasopharyngeal swab or an oropharyngeal swab. Samples also can be samples that have been enriched for particular types of molecules, e.g., DNA or RNAs.

The sample can be obtained directly from a subject, derived from a subject, or derived from samples obtained from a subject, such as cultured cells derived from a biological fluid or tissue sample. The sample can be a fresh sample. The fresh sample can be fixed after removal from the subject with any known fixatives (e.g. formalin, Zenker's fixative, or B-5 fixative). The sample can also be archived samples, such as frozen samples, cryopreserved samples, of cells obtained directly from a subject or of cells derived from cells obtained from a subject.

Samples can be obtained from a subject by any means including, but not limited to, biopsy, needle aspirate, scraping, surgical incision, venipuncture, or other means known in the art.

The sample can be obtained from the subject in a single procedure. The sample can be obtained from the subject repeatedly over a period of time. For example, once a day, once a week, monthly, biannually, or annually. Obtaining numerous samples over a period of time can be used to profile and/or monitor target nucleic acids. The sample can be obtained from the same location or a different location.

The sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms, a plant or a plant cell, an algal cell, a fungal cell, an animal cell, tissue, or organ, a cell, tissue, or organ from an invertebrate animal, a cell, tissue, fluid, or organ from a vertebrate animal, a cell, tissue, fluid, or organ from a mammal (e.g., a human or a non-human primate). Suitable sample sources also include nematodes, protozoans, and the like. Suitable sample sources include parasites such as helminths, and malarial parasites.

Where the organism is a plant, suitable sources include xylem, the phloem, the cambium layer, leaves, or roots. Where the organism is an animal, suitable sources include a sample from the lung, liver, heart, kidney, brain, spleen, skin, fetal tissue, or a particular cell type (e.g., neuronal cells, epithelial cells, endothelial cells, astrocytes, macrophages, glial cells, islet cells, T lymphocytes, or B lymphocytes).

### H. Measuring a Fluorescence Signal

The methods disclosed herein include a step of measuring a fluorescence signal as an indicator of whether the target nucleic acid is present in the sample. The presence of a fluorescence signal indicates the presence of the target nucleic acid in the sample. The absence of a fluorescence signal can indicate the target nucleic acid is not present in the sample. Alternatively, the absence of a fluorescence signal can indicate that the target nucleic acid is present in an amount that is not sufficient to be detectable according to the methods disclosed herein.

Any fluorescent label can be utilized. Examples of fluorescent labels include, but are not limited to, an Alexa Fluor® dye, an ATTO dye (e.g., ATTO 390, ATTO 425, ATTO 465, ATTO488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), fluorescein amidite (FAM), tetramethylrhodamine (TRITC), Texas Red, Oregon Green, Pacific Blue, Pacific Green, Pacific Orange, quantum dots, and a tethered fluorescent protein.

The fluorescence signal can be produced by a fluorescence-emitting dye pair. For example, a fluorescence resonance energy transfer (FRET) pair or a quencher/fluor pair, or both. The term “fluorescence-emitting dye pair,” as used herein encompasses both a “fluorescence resonance energy transfer (FRET) pair” and a “quencher/fluor pair” FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table A. FRET pairs provided in U.S. Pat. No. 10,253,365 are incorporate by reference herein in their entirety. The FRET pair can be a 5′ 6-FAM and 3IABKFQ (Iowa Black®-FQ) (Integrated DNA Technologies, Inc., Coralville, IA).

TABLE A

| Donor | Acceptor |
|---|---|
| Tryptophan | Dansyl |
| IAEDANS (1) | DDPM (2) |
| BFP | DsRFP |
| Dansyl | Fluorescein |
| | Isothiocyanate (FITC) |
| Dansyl | Octadecylrhodamine |
| Cyan fluorescent | Green fluorescent protein |
| protein (CFP) | (GFP) |
| CF(3) | Texas Red |
| Fluorescein | Tetramethylrhodamine |
| Cy3 | Cy5 |
| BODIPY FL (4) | Yellow fluorescent protein |
| | (YFP) |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

The detectable signal can be produced when the labeled detector is cleaved.

The fluorescence signal can be measured using any suitable technique. The type of fluorescence reporter used in the method can determine the technique employed for measuring the fluorescence signal. For example, the fluorescence signal can be measured using a fluorescent optical detection system including but not limited to fluorometers, spectrophotometers, microplate readers, photodetectors, and light dependent resistors, Western blot, gel electrophoresis, microscopy, a camera, a fluorometer, or a lamp (for example, a Xenon flash lamp, a halogen lamp, or a light emitting diode). The fluorescence signal can be measured visually by the intensities of the fluorescence signal.

The sample can be contacted with the CRISPR/Cas complex, one or more nucleases, one or more oligonucleotides, and a fluorescence reporter for about 30 seconds, about 1 minute, about 2 minutes, about 5 minutes, about 10 minutes, about 20 minutes, about 30 minutes, about 40 minutes, about 50 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 12 hours, or longer prior to measuring the fluorescence signal. For example, the sample can be contacted for 20 minutes prior to measuring the fluorescence signal. For example, the sample can be contacted for 30 seconds or less prior to measuring the fluorescence signal. For example, the sample can be contacted for 1 minute or less prior to measuring the fluorescence signal. For example, the sample can be contacted for 30 minutes or less prior to measuring the fluorescence signal. For example, the sample can be contacted for 40 minutes prior to measuring the fluorescence signal. For example, the sample can be contacted for 1 hour prior to measuring the fluorescence signal. For example, the sample can be contacted for 2 hours prior to measuring the fluorescence signal.

The methods disclosed herein can detect a target nucleic acid in a sample with a high degree of sensitivity. The methods disclosed herein can be used to detect a target nucleic acid in a sample comprising one or more target nucleic acids. The methods disclosed herein can exhibit an attomolar (aM) sensitivity of detection, a femtomolar (fM) sensitivity of detection, or a picomolar (pM) sensitivity of detection.

The methods disclosed herein can detect a target nucleic acid in a sample at a concentration of 10 nM or less, about 5 nM or less, about 1 nM or less, about 0.5 nM or less, about 0.1 nM or less, about 0.01 nM or less, about 0.0005 nM or less. In embodiments, the methods disclosed herein can detect a target nucleic acid in a sample at a concentration of about 10 pM or less, about 5 pM or less, about 2 pM or less, about 1 pM or less. In embodiments, the methods disclosed herein can detect a target nucleic acid of about 500 fM or less, about 200 fM or less, about 100 fM or less, or about 50 fM or less. In embodiments, the methods disclosed herein can detect a target nucleic acid of about 500 aM or less, about 250 aM or less, about 100 aM or less, about 50 aM or less, about 25 aM, about 10 aM or less, about 5 aM or less, about 2 aM or less, about 1 aM or less.

In embodiments, the methods disclosed herein can detect a target nucleic acid in the range from 2 aM to 1 nM. For example, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM.

### I. Methods of Use

Provided herein are methods for identifying a subject having or suspected of having a disease. The methods disclosed herein can be applied in any situation that requires the detection of a DNA or RNA target. The methods of identifying a subject having or suspected of having a disease may comprise providing to a sample (i) a CRISPR/Cas complex that comprises an effector nuclease and a guide RNA, (ii) one or more nucleases, (iii) one or more oligonucleotides, and (iv) a fluorescence reporter. The method may further comprise measuring a fluorescence signal emitted from the fluorescence reporter. The presence of a fluorescence signal indicates the presence of disease (e.g., a target nucleic acid).

Exemplary diseases detectable by the methods described herein include, but are not limited to a cancer, an autoimmune disease, an infection, or a sexually transmitted disease.

The infection can be caused by a virus, a bacteria, a fungus, a parasite, or a protozoa. The source of the target nucleic acid is described above.

The methods disclosed herein can detect single nucleotide polymorphisms in genes or gene variants. The methods of detecting a single nucleotide polymorphism in a gene or gene variant may comprise providing to a sample (i) a CRISPR/Cas complex that comprises an effector nuclease and a guide RNA, (ii) one or more nucleases, (iii) one or more oligonucleotides, and (iv) a fluorescence reporter. The method may further comprise measuring a fluorescence signal emitted from the fluorescence reporter. The presence of a fluorescence signal indicates the presence of a single nucleotide polymorphism.

### J. Kits

Also disclosed herein are kits comprising a CRISPR/Cas complex, one or more oligonucleotides, one or more nucleases, and a fluorescence reporter.

The kits can be used for a variety of applications. A preferred application is for the identification and/or detection of a target nucleic acid in a sample. One skilled in the art will recognize components of kits suitable for carrying out a method (or methods) of the present disclosure. For example, a kit may include one or more containers, each of which is suitable for containing one or more reagents or other means for detecting a target nucleic acid, instructions for detecting a target nucleic acid using the kit, and optionally instructions for carrying out one or more of the methods descried herein.

In some instances, the kit may also include one or more vials, tubes, bottles, dispensers, and the like, which are capable of holding one or more reagents needed to practice the present disclosure.

Instructions for the kits of the present disclosure may be affixed to packaging material, included as a package insert, and/or identified by a link to a website. While the instructions are typically written or printed materials, they are not limited to such. Any medium capable of storing such instructions and communicating them to an end user is contemplated by the present disclosure. Such media includes, but is not limited to, electronic storage media (e.g., magnetic discs, tapes, cartridges, chips), optical media (e.g., CD ROM), and the like. As used herein, the term “instructions” can include the address of an Internet site that provides the instructions. An example of this can include a kit that provides a web address where the instructions can be viewed and/or from which the instructions can be downloaded. In other instances, kits of the present disclosure may comprise one or more computer programs that may be used in practicing the methods of the present disclosure. An example of this can include a kit that provides a web address where the instructions can be viewed and/or from which the instructions can be downloaded. In other instances, kits of the present disclosure may comprise one or more computer programs that may be used in practicing the methods of the present disclosure. For example, a computer program may be provided that takes the output from a microplate reader or a fluorescence spectrophotometer and prepares a calibration curve from the optical density observed in the wells and compares these densitometric or other quantitative readings to the optical density or other quantitative readings in wells.

The kit can be used to detect any suitable target nucleic acid. For example, the kit may be used to detect a viral nucleic acid. For example, the kit may be used to detect a bacterial nucleic acid. For example, the kit may be used to detect a fungal nucleic acid. For example, the kit may be used to detect a parasitic nucleic acid.

The kit of the present disclosure can include a positive control guide RNA or a positive control target nucleic acid. The positive control guide RNA can comprise a nucleotide sequence that hybridizes to the control target nucleic acid. In embodiments, the positive control target nucleic acid can be DNA or RNA.

The kit of the present disclosure can be included in a cartridge or a device.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain methods in detail, the same will be more clearly understood by reference to the following examples, which are introduced for illustration only and not intended to be limiting.

### EXAMPLES

### Example 1.1: Cas12p/Csx1 Activity Assay

The Cas12p/Csx1 activity assay was performed using the conditions described in Table 1.

TABLE 1
Conditions

| Label | Condition |
|---|---|
| 1 | Csx1 + Cas12p + RNAse Alert + RdRP Activator |
| 2 | Cas12p + RNAse Alert + RdRP Activator |
| 3 | Csx1 + RNAse Alert + RdRP Activator |
| 4 | Csx1 + Cas12p + RNAse Alert |
| 5 | Cas12p + RNAse Alert |
| 6 | Csx1 + RNAse Alert |
| + | Positive control: Cas12p + FAM-Q + RdRP Activator |
| − | Negative control: Cas12p + FAM-Q |

The reagents used in the assay include: Csx1 p2 (stock 280 nM), Cas12p (stock 2 μM), RdRP activator (stock 2 nM), sgRNA RdRP (stock 2 μM), buffer 2.1 (stock 10×), TTATT FAM-Q (stock 100 μM), RNAse Alert Substrate, IDT (25 pmol each tube), and nuclease free water.

The CRISPR/Cas complex reagents were prepared as outlined in Table 2 and incubated for 20 minutes at room temperature. The Fam-Q mixture was prepared as shown in Table 3. Then, the RNAseAlert Mix was prepared according to Table 4.

TABLE 2
CRISPR Mix

| Reagent | Stock CC | Final cc | Vol/Rx | | Mix A (x6) | |
|---|---|---|---|---|---|---|
| NF water | — | — | 12.7 | μl | 76.2 | μl |
| Buffer 2.1 | 10X | 1X | 2 | μl | 12 | μl |
| NEB | | | | | | |
| sgRNA | 2 μM | 75 nM | 1.5 | μl | 9 | μl |
| Cas12p | 2 μM | 75 nM | 1.5 | μl | 9 | μl |
| Final vol. | — | — | 17.7 | μl | 106.2 | μl |

TABLE 3
FAM-Q Mix

| Reagent | Stock CC | Final cc | Vol/Rx | | Mix A (x6) | |
|---|---|---|---|---|---|---|
| NF water | — | — | 18.05 | μl | 36.1 | μl |
| Buffer 2.1 | 10X | 1X | 2 | μl | 4 | μl |
| NEB | | | | | | |
| Final vol. | — | — | 20.3 | μl | 40.6 | μl |

TABLE 4
RNAseAlert Mix

| Reagent | Stock CC | Final cc | Vol/Rx | | Mix A (x6) | |
|---|---|---|---|---|---|---|
| NF water | — | — | 12.7 | μl | 50.8 | μl |
| Buffer 2.1 | 10X | 1X | 2 | μl | 8 | μl |
| NEB | | | | | | |
| RNAseAlert | 25 pmol | 12.5 pmol | 3 | μl | 12 | μl |
| Final vol. | — | — | 17.7 | μl | 70.8 | μl |

The CRISPR mix was divided as outlined in Table 5. The RNAseAlert Mix was divided as outlined in Table 6.

TABLE 5
CRISPR mix

| Mix | Volume | Tube |
|---|---|---|
| (A) CRISPR Mix | 35.4 μl | (B) FAM-Q Mix |
| | 70.8 μl | (C) RNAseAlert Mix |

TABLE 6
RNAseAlert Mix

| Mix | Volume | Tube |
|---|---|---|
| (C) RNAseAlert Mix | 70.8 μl | (D) Cas12p/Csx1 Mix |
| | 70.8 μl | (C) Cas12p Mix |

The following reagents were added to the corresponding mix (Table 7).

TABLE 7
Reagents added to mixture

| Mix | Stock cc | Final cc | Vol/Rx | Rx = 2 | Tube |
|---|---|---|---|---|---|
| Csx1 | 280 nM | 18 nM | 2.6 μl | 5.2 μl | (D) Cas12p/ |
| | | | | | Csx1 Mix |
| NF Water | — | — | 2.6 μl | 5.2 μl | (E) Cas12p |
| | | | | | Mix |

Next, the mixture as outlined in Table 8 was prepared.

TABLE 8
(F) Csx1 Mix

| Reagent | Stock cc | Final cc | | Vol/Rx | | Mix F (x2) | |
|---|---|---|---|---|---|---|---|
| NF water | — | — | | 27.4 | μl | 54.8 | μl |
| Buffer 2.1 | 10 X | 1X | | 4 | μl | 8 | μl |
| NEB | | | | | | | |
| RNAse Alert | 25 pmol each | 16 | pmol | 4 | μl | 8 | μl |
| | tube | | | | | | |
| Csx1 | 280 nM | 18 | nM | 2.6 | μl | 5.2 | μl |

| | | | | | | |
|---|---|---|---|---|---|---|
| Final Vol. | — | — | 38 | μl | 76 | μl |

Each mixture was then divided as outlined in Table 9.

TABLE 9
Divided mixture

| Mix | Volume | Tubes |
|---|---|---|
| (D) Cas12p/Csx1 Mix | 38 μl | 1 and 4 |
| (E) Cas12p Mix | 38 μl | 2 and 5 |
| (F) Csx1 Mix | 38 μl | 3 and 6 |
| (B) FAM-1 Mix | 38 μl | Positive and Negative Control |

The reagents outlined in Table 10 were added to the mixtures of Table 9.

| Reagent | Stock cc | | Final cc | | Vol/Rx | Tube |
|---|---|---|---|---|---|---|
| Nuclease- | — | | | | 2 μl | 4, 5, 6, and |
| free water | | | | | | Negative control |
| RdRP activator | 2 | nM | 100 | pM | 2 μl | 1, 2, and 3 |
| | 20 | nM | 1 | nM | 2 μl | Positive control |

The mixtures were incubated at 42° C. and read every 5 minutes in a plate reader.

### Example 1.2: NucC Activity Assay with M13 Plasmid

The objective of this experiment was to determine whether the NucC enzyme cleaves both double-stranded DNA and single-stranded DNA by incubating the enzyme with its cyclic activator (c-triAMP) and the dsM13 or ssM13 plasmid.

The conditions that were tested are outlined in Table 11 below.

TABLE 11
Conditions tested.

| Label | Condition |
|---|---|
| 1 | NucC + c(triAMP) + Buffer 2.1 + dsM13 |
| | (M13mp18 RF I plasmid) |
| 2 | NucC + Buffer 2.1 + dsM13 (M13mp18 RF I plasmid) |
| 3 | Buffer 2.1 + dsM13 (M13mp18 RF I plasmid) |
| 4 | NucC + c(triAMP) + Buffer 2.1 + ssM13 |
| | (M13mp18 single-stranded plasmid) |
| 5 | NucC + Buffer 2.1 + ssM13 (M13mp18 |
| | single-stranded plasmid) |
| 6 | Buffer 2.1 + ssM13 (M13mp18 single-stranded plasmid) |

A first NucC Mix (dsM13) was prepared as outlined in Table 12 below.

TABLE 12
NucC Mix (dsM13) mixture

| | | | | Tube | Tube | Tube |
|---|---|---|---|---|---|---|
| Reagent | Stock cc | Final cc | Vol/Rx | 1 (x2) | 2 (x2) | 3 (x2) |
| NF water | — | — | *** | 38 μl | 38.5 μl | 39 μl |
| Buffer 2.1 | 10X | 1X | 2.5 μl | 5 μl | 5 μl | 5 μl |
| NucC enzyme | 1 μM | 10 nM | 0.25 μl | 0.5 μl | 0.5 μl | — |
| c(triAMP) | 1 μM | 10 nM | 0.25 μl | 0.5 μl | — | — |
| dsM13 plasmid | 100 ng/μl | 300 ng | 3 μl | 6 μl | 6 μl | 6 μl |
| Final vol. | — | — | 25 μl | 50 μl | 50 μl | 50 μl |

A second NucC Mix (ssM13) was prepared as outlined in Table 13 below.

TABLE 13
NucC Mix (ssM13) mixture

| | | | | Tube 4 | Tube | Tube |
|---|---|---|---|---|---|---|
| Reagent | Stock cc | Final cc | Vol/Rx | (x2) | 5 (x2) | 6 (x2) |
| NF water | — | — | *** | 40 μl | 40.5 μl | 41 μl |
| Buffer 2.1 | 10X | 1X | 2.5 μl | 5 μl | 5 μl | 5 μl |
| NucC enzyme | 1 μM | 10 nM | 0.25 μl | 0.5 μl | 0.5 μl | — |
| c(triAMP) | 1 μM | 10 nM | 0.25 μl | 0.5 μl | — | — |
| ssM13 plasmid | 250 ng/μl | 500 ng | 2 μl | 4 μl | 4 μl | 4 μl |
| Final vol. | — | — | 25 μl | 50 μl | 50 μl | 50 μl |

Each mixture described in Table 12 and Table 13 was divided into two PCT tubes. The mixture was then incubated at 37° C. for 2:30 hours. Next, the samples were run on a 1% agarose gel.

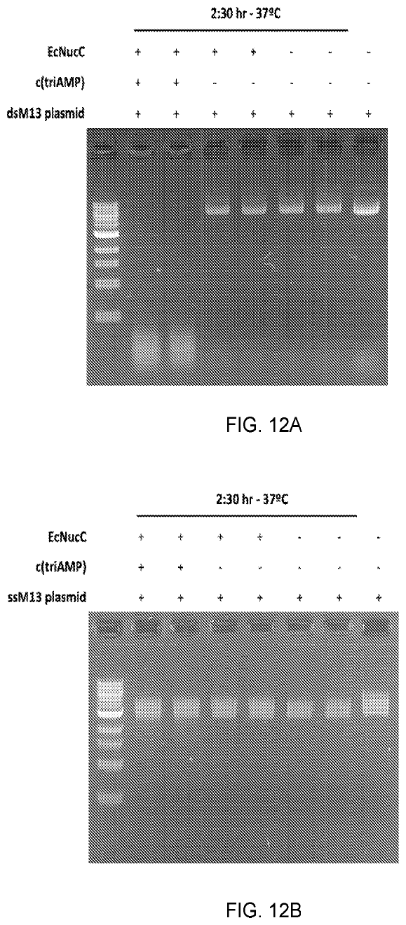

The NucC enzyme cleaved double-stranded DNA, but not single stranded RNA. See, FIGS. 12A and 12B.

### Example 1.3: NucC Activity Assay with M13 Plasmid

The objective of this experiment was to achieve activation of the NucC enzyme by incubating this enzyme with its cyclic (c-triAMP) and linear (triAMP)>P activator and the dsM13 plasmid.

The conditions that were tested are outlined in Table 14 below.

TABLE 14
Conditions tested

| Label | Condition |
|---|---|
| 1 | NucC + c(triAMP) + Buffer 2.1 + dsM13 |
| | (M13mp18 RF I plasmid) |
| 2 | NucC + (triAMP) > P + Buffer 2.1 + dsM13 |
| | (M13mp18 RF I plasmid) |
| 3 | NucC + Buffer 2.1 + dsM13 (M13mp18 |
| | RF I plasmid) |
| 4 | Buffer 2.1 + dsM13 (M13mp18 RF I plasmid) |

A NucC Mix (dsM13) was prepared as outlined in Table 15 below.

TABLE 15
NucC Mix (dsM13) mixture

| | | | | Tube | Tube | Tube | Tube |
|---|---|---|---|---|---|---|---|
| | | Final | Vol/ | 1 | 2 | 3 | 4 |
| Reagent | Stock cc | cc | Rx | (x2) | (x2) | (x2) | (x2) |
| NF water | — | — | *** | 36 μl | 36 μl | 36.5 μl | 37 μl |
| Buffer 2.1 | 10X | 1X | 2.5 μl | 5 μl | 5 μl | 5 μl | 5 μl |
| NucC | 1 μM | 10 nM | 0.25 μl | 0.5 μl | 0.5 μl | 0.5 μl | — |
| enzyme | | | | | | | |
| c(triAMP) | 1 μM | 10 nM | 0.25 μl | 0.5 μl | — | — | — |
| (triAMP) > | 1 μM | 10 nM | | — | 0.5 μl | — | — |
| P | | | | | | | |
| dsM13 | 100 μg/ml | 400 ng | 4 μl | 8 μl | 8 μl | 8 μl | 8 μl |
| plasmid | | | | | | | |
| Final vol. | — | — | 25 μl | 50 μl | 50 μl | 50 μl | 50 μl |

The mixture described in Table 15 was divided into two PCT tubes. The mixture was then incubated at 37° C. for 4 hours. Next, the samples were run on a 1.5% agarose gel.

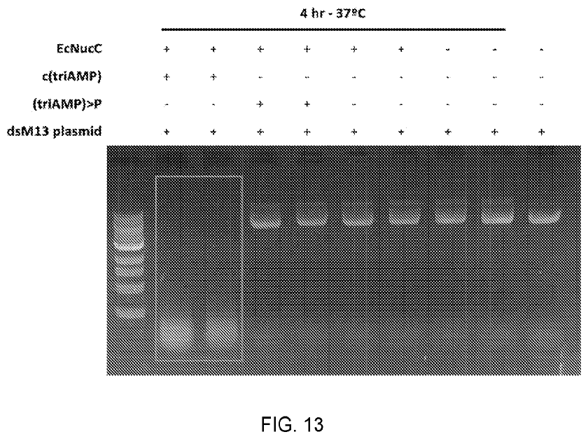

Activation of the NucC enzyme with its cyclic RNA activator (c-triAMP) was achieved. As shown in FIG. 13, complete degradation of the dsM13 plasmid was observed when the NucC enzyme was incubated in the presence of c-triAMP.

### Example 1.4: Activation of SyCsx1, PfuCsx1, and TtCsm6 by Linear and Cyclic Activators

The objective of these experiments was to assess activation of SyCsx1, PfuCsx1, and TtCsm6 by incubating the nucleases with cyclic or linear activators.

Varying concentrations of nuclease (SyCsx1, PfuCsx1, or TtCsm6) in reaction buffer (2.1 NEB Buffer-50 mM NaCl, 10 mM Tris-HCl, 10 mM MgCl₂, 100 μg/ml BSA) with 1 U/μl murine RNase inhibitor (NEB) and 600 nM FAM-Q reporter (IDT) was mixed with varying concentrations of cA3, cA4, cA6, rA3>P or rA4>P (Biolog or IDT). Negative control was water free of activator. Measurements were taken at 37° C. every 1 min in a Tecan Infinite 200 PRO plate reader (λex, 485 nm; λem, 535 nm). Examples 1.5-1.6 followed similar protocols.

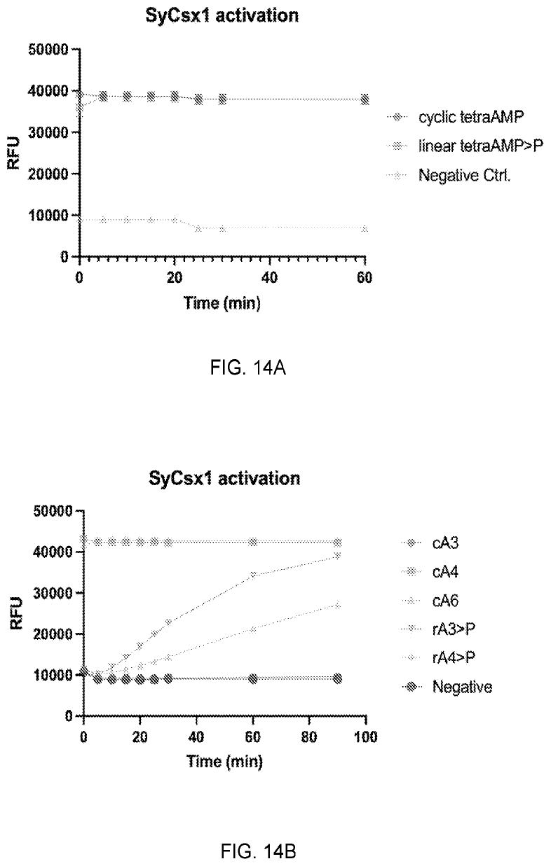

FIG. 14A shows time courses of fluorescence, in relative fluorescence units (RFU), for the activation of SyCsx1 by cA4 and rA4>P. The reporter used was FAM-Q polyC (ssRNA reporter) and the SyCsx1 concentration was 100 nM. SyCsx1 was activated by both cA4 and rA4>P. Negative control remained constant.

FIG. 14B shows time courses of fluorescence in RFU for the activation of SyCsx1 by cyclic triAMP (cA3), cA4, cyclic hexaAMP (cA6), linear triAMP>P (rA3>P), and rA4>P. The reporter used was FAM-Q polyC (ssRNA reporter) and the SyCsx1 concentration was 100 nM. SyCsx1 was activated by cA4, cA6, rA3>P, and rA4>P. The most rapid activations (shortest times until maximum fluorescence) were achieved by cA4 and rA4>P. Negative control remained constant.

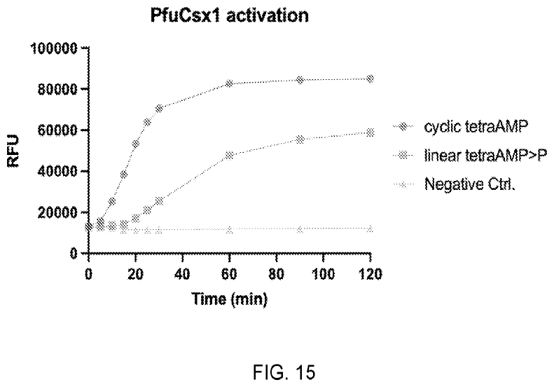

FIG. 15 shows time courses of fluorescence in RFU for the activation of PfuCsx1 by cA4 and rA4>P. The reporter used was FAM-Q polyA (ssRNA reporter) and the PfuCsx1 concentration was 100 nM. Both cA4 and rA4>P activated PfuCsx1. Negative control remained constant.

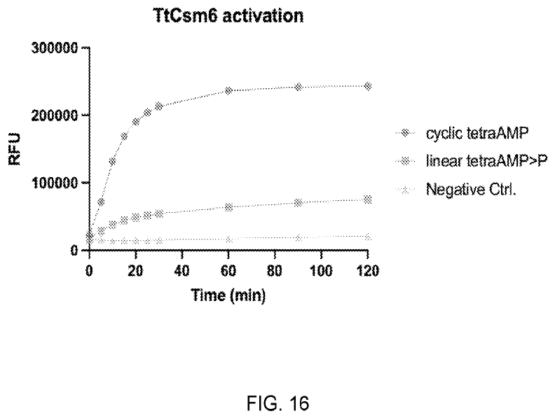

FIG. 16 shows time courses of fluorescence in RFU for the activation of TtCsm6 by cA4 and rA4>P. The reporter used was RNAse Alert (IDT) and the TtCsm6 concentration was 100 nM. Both cA4 and rA4>P activated TtCsm6. Negative control remained constant.

### Example 1.5: Limit of Detection (LoD) Assays for SyCsx1, PfuCsx1, and TtCsm6 Activated by rA4>P

The objective of these experiments was to determine the detection limit of SyCsx1, PfuCsx1, and TtCsm6 incubated with various concentrations of linear activator rA4>P. The protocol set forth under Example 1.4 was generally followed.

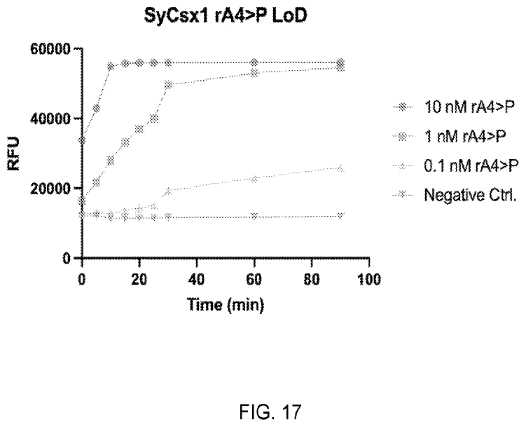

FIG. 17 shows time courses of fluorescence for activation of SyCsx1 by 0.1 nM, 1 nM, or 10 nM rA4>P. The reporter used was FAM-Q polyC (ssRNA reporter) and the SyCsx1 concentration was 10 nM. SyCsx1 was activated by as low as 0.1 nM (100 pM) of rA4>P. Negative control remained constant.

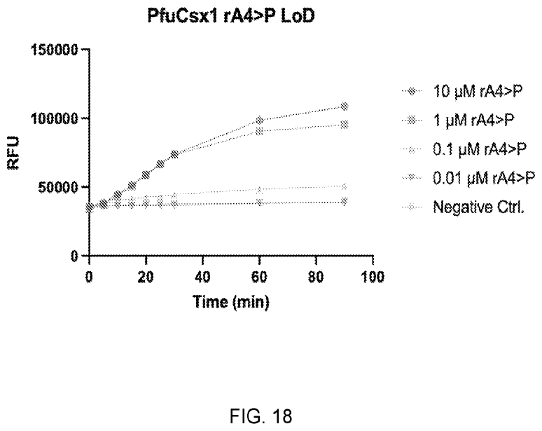

FIG. 18 shows time courses of fluorescence for activation of PfuCsx1 by 0.01 μM, 0.1 μM, 1 μM, or 10 μM rA4>P. The reporter used was FAM-Q polyA (ssRNA reporter) and the PfuCsx1 concentration was 100 nM. Pfucsx1 was activated by 1 μM (or higher) rA4>P. Negative control remained constant.

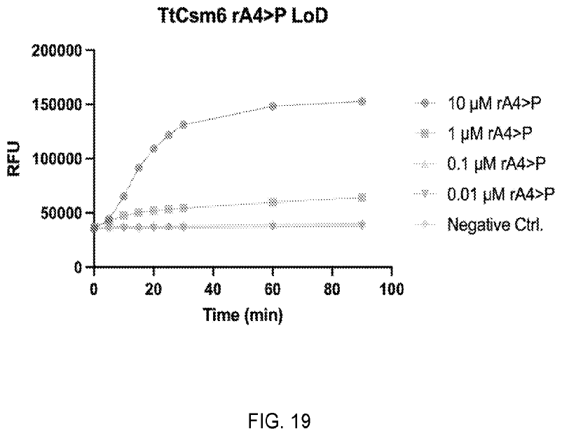

FIG. 19 shows time courses of fluorescence for activation of TtCsm6 by 0.01 μM, 0.1 μM, 1 μM, or 10 μM rA4>P. The reporter used was FAM-Q polyC (ssRNA reporter) and the TtCsm6 concentration was 100 nM. TtCsm6 was activated by 1 μM (or higher) of rA4>P. Negative control remained constant.

### Example 1.6: Reporter Preference Assays for SyCsx1, PfuCsx1, and TtCsm6 Activated by cA4

The objective of these experiments was to determine the preferred reporters for SyCsx1, PfuCsx1, and TtCsm6 incubated with cyclic activator cA4. The protocol set forth under Example 1.4 was generally followed.

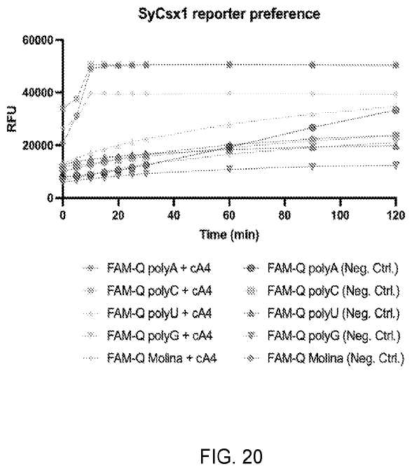

FIG. 20 shows time courses of fluorescence for reporters FAM-Q polyA, FAM-Q polyC, FAM-Q polyU, FAM-Q polyG, and FAM-Q UCU (5′-/56 FAM/rArUrGrUrCrCrCrCrUrGrArA/3IABKFQ/-3′ or 6-carboxyfluorescein/SEQ ID NO: 22/Iowa Black® FQ), upon cleavage thereof by SyCsx1 activated by cA4. The SyCsx1 concentration was 100 nM. The cA4 concentration was 10 μM. The best reporters were FAM-Q polyC, FAM-Q polyA and FAM-Q UCU. Negative controls (reporters without cA4) remained constant.

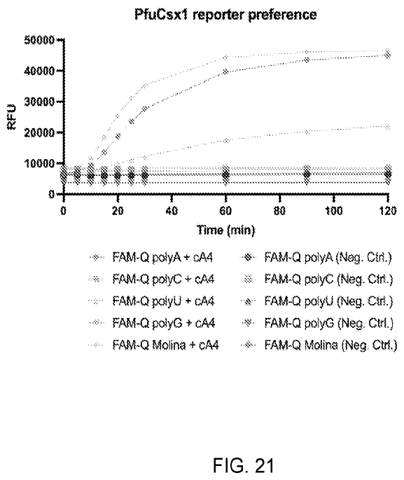

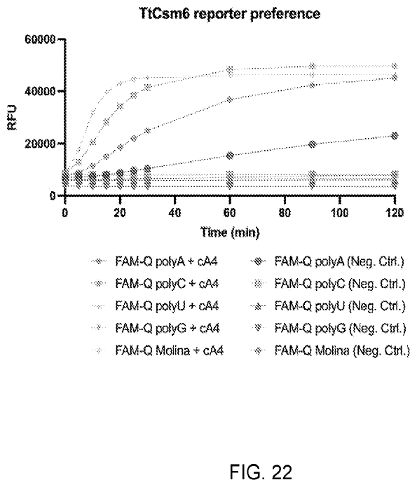

FIG. 21 shows time courses of fluorescence for reporters FAM-Q polyA, FAM-Q polyC, FAM-Q polyU, FAM-Q polyG, and FAM-Q UCU, upon cleavage thereof by PfuCsx1activated by cA4. The PfuCsx1concentration was 100 nM. The cA4 concentration was 10 μM. The best reporters were FAM-Q UCU and FAM-Q polyA. Negative controls (reporters without cA4) remained constant.

FIG. 22 shows time courses of fluorescence for reporters FAM-Q polyA, FAM-Q polyC, FAM-Q polyU, FAM-Q polyG, and FAM-Q UCU, upon cleavage thereof by TtCsm6 activated by cA4. The TtCsm6 concentration was 100 nM. The cA4 concentration was 10 μM. The best reporters were FAM-Q UCU and FAM-Q polyC. Negative controls (reporters without cA4) remained constant.

### Example 1.7: Cascaded CRISPR Assays for Detection of SARS RNA

The objective of these experiments was to determine the limit of detection (LoD) of SARS RNA by Cas-Nuclease pairs LbuCas13a/SyCsx1 and LbuCas13a/TtCsm6.

Cas-Nuclease reactions contained 75 nM Cas enzyme, 75 nM crRNA, varying concentrations of nuclease (SyCsx1, PfuCsx1 TtCsm6 or others), extracted RNA SARS samples (PCR detection cycle threshold (Ct) varying from 15 to 27, with large numbers indicative of lower RNA levels in sample, or SARS RNA-negative control) and 600 nM FAM-Q reporter, with either pre-second messenger or nuclease-free water added (negative control). The reactions were performed at 37° C. in 2.1 buffer (NEB) containing 50 mM NaCl, 10 mM Tris-HCl, 10 mM MgCl2, 100 ug/ml BSA. The Cas-crRNA complex was assembled at a concentration for 20 min at room temperature. Fluorescence measurements were taken at 37° C. every minute in a Tecan Infinite 200 PRO plate reader (λex, 485 nm; λem, 535 nm).

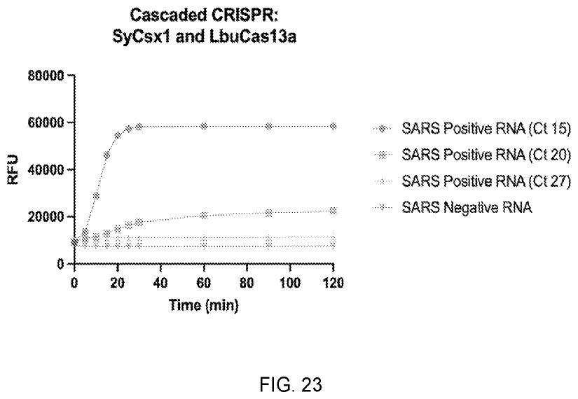

FIG. 23 shows time courses of fluorescence by a system containing SyCsx1 in combination with LbuCas13a. The pre-second messenger used was rA4(rU5) at 2 μM. The reporter used was FAM-Q polyC (ssRNA reporter), the SyCsx1 concentration was 25 nM, and the LbuCas13a complex concentration was 75 nM. SARS positive extracted RNA samples were detected in samples with Ct 15 and Ct 20.

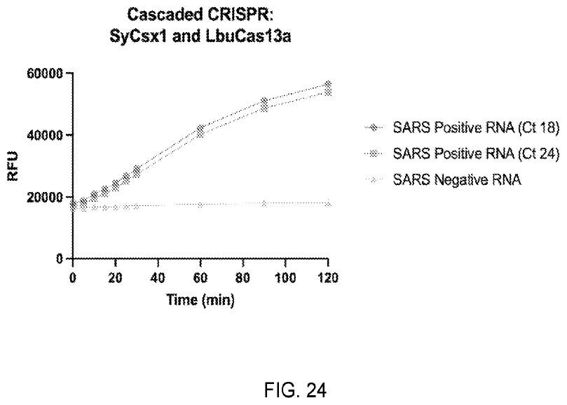

FIG. 24 shows time courses of fluorescence by a system containing SyCsx1 in combination with LbuCas13a. A combination of pre-second messengers was used, 5 μM rA4(rU5) and 1 μM rA4(1-2*)(rC5). The reporter used was FAM-Q polyC (ssRNA reporter), the SyCsx1 concentration was 20 nM, and the LbuCas13a complex concentration was 150 nM. SARS positive extracted RNA samples were detected in samples with Ct 18 and Ct 24.

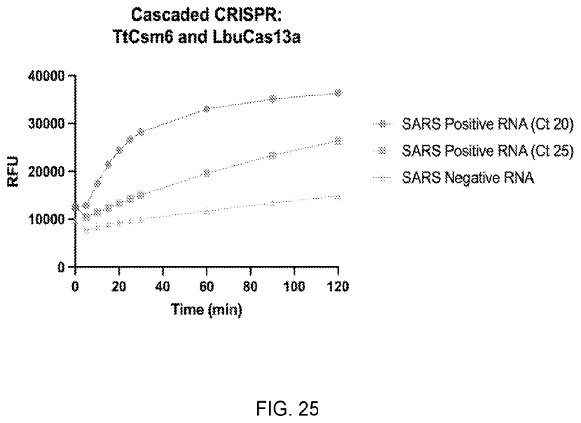

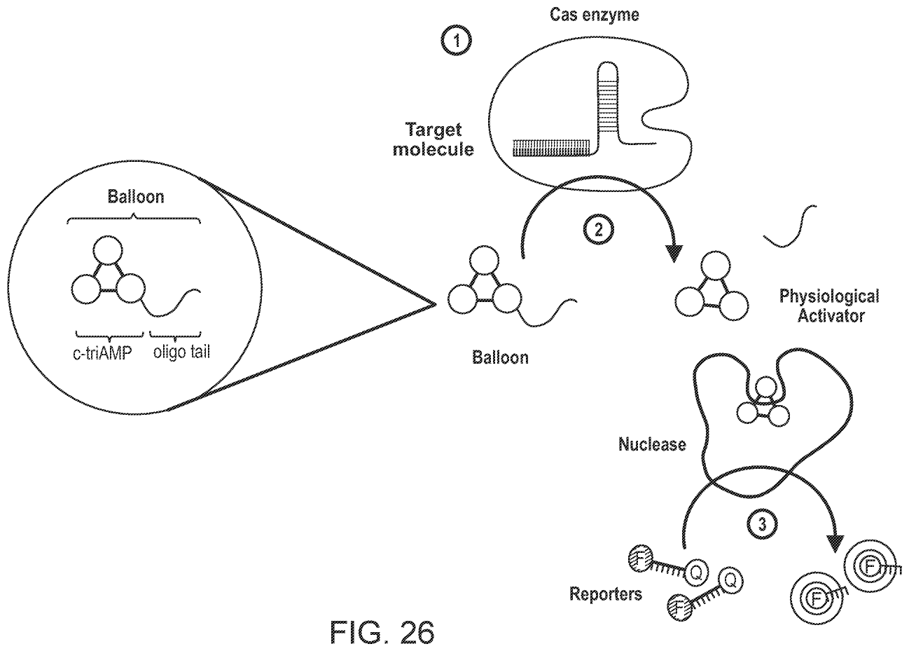

FIG. 25 shows time courses of fluorescence by a system containing TtCsm6 in combination with LbuCas13a. The pre-second messenger used was rA4(rU5) at 2 μM. The reporter used was FAM-Q UCU (ssRNA reporter), the TtCsm6 concentration was 100 nM, and the LbuCas13a complex concentration was 75 nM. SARS positive extracted RNA samples were detected in samples with Ct 20 and Ct 25.

### SEQUENCE LISTING

| SEQ ID NO: | Description | Sequence |
|---|---|---|
| SEQ ID NO: 1 | Amino acid | AAHHHHHHSSGLVPRGSHMKCLFYIAGDVSNYSIVNYELNGQTQN |
| | sequence of | TFFAAHALYNLFKPDKVIALIPDSLVKDNVSDEECYKNLVINRAK |
| | Csx1 | ELNFAGMEEFMNKVEIRKIPNVGIASAIQCENGAPKKEKNKEGRE |
| | | VLKRLPYNEKRSPIFIFNAIYAIFKDEACDEYLVDLTHGTNVLVS |
| | | IGMNVGALFNAKFYSAPVMGMPGKDSIVNIVELTDVVQATNDSLM |
| | | IRSSIENLDERYFKDYSAKLSRLNPTIFEEEEKKVLTRVKGTDVN |
| | | VVINFLWNIRNGFTVNAVKSMNELKNIINQLEEDLEKLKSFYKNW |
| | | EEHKNFQGETLLVLSDLDSTLKVKDLLIEGNDLEKLNYLLDLYIK |
| | | ASIYDKALSLARELPVAICLNKVGGGMFDDKNEKYKHCNEIVTSY |
| | | LRLRYSGLMEFRNTLMHGGLSTDMKPNVDKDGNITPGKIVTKNKI |
| | | EDFVKRELRNYFDKIVNFLSSA |
| SEQ ID NO: 2 | Amino acid | MSDWSLSQLFASLHEDIQLRLGTARKAFQHPGAKGDASEGVWIEM |
| | sequence of | LDTYLPKRYQAANAFVVDSLGNFSDQIDVVVFDRQYSPFIFKFNE |
| | NucC wild | QIIVPAESVYAVFEAKQSASADLVAYAQRKVASVRRLHRTSLPIP |
| | sequence | HAGGTYPAKPLIPILGGLLTFESDWSPALGMSFDKALNGDLSDGR |
| | | LDMGCVASHGHFYFNNIDSKFNFEHGNKPATAFLFRLIAQLQFSG |
| | | TVPMIDIDAYGKWLAN |
| SEQ ID NO: 3 | Amino acid | MQAPVYLCLLGNDPAPAYLGLKVVEREAGRVAKAVFYSFPAWNEE |
| | sequence of | YGKKRQAFFRLLSEKGVLYEERPLEKGLEEAEAREVWVNLTGGAK |
| | Can1 wild | YWAVRFLGHWRRPGARVFLVEGHRALEAPRALFLWPREEERSLEA |
| | sequence | EALTLEEYARLYLEPLGEAWERVSPPGAFPPGAQAARLPGREGGV |
| | | FVVHRGLPYWYWVRPHLGGEAKDMSRKALSAFSGEAKRLGGQLCL |
| | | PVVPYHKAHLRSRHPKERENVFARWRAWAREYGVFLVDPGRPLEE |
| | | EVASLIKGKASKKALPLPQEGPLLLALVSEQAVPLYAAYLHAGPR |
| | | EVYLLTTPEMESRLRWAEAFFRGKGVRVHRSFLSGPWALREVRDL |
| | | LAPVVEEALRRGHPVHANLNSGTTAMALGLYLALRDGARAHYLDG |
| | | DRLLLLDGGEAEVPWEEGRPEDLLALRGYRFEEEYPDARPDPGLL |
| | | ALAEEILRRWDEVQTSWEASPLVRRFLKFWKKRFGQAFPPKRLSR |
| | | LKGLPLEYAVYSHLNAHLAPKGGQARMGGHLVPLGGNEALAPQST |
| | | EVDGVFFHRGALWFVECKPTDEGLRERAPIMAELVRSVGGVEARG |
| | | LMVARRWRGAPPPASPNLVYMALEGGEGVGVYRFPEELEKALSRN |
| | | PAPRRG |
| SEQ ID NO: 4 | Amino acid | MSASLLEKQSTGGAIARVGFGYQDAFVLRSLPLWLSQSAFSHIVS |
| | sequence of | EALSDIEVCYFSSEKSLHVMYEAKNHSLTATEFWDEIRRFKSLFD |
| | AbCap4 wild | THPKNFIWFNLVCPSYNTAISPLISKIDRLRGVGSSYDDDSSVSV |
| | sequence | NGRSEYLDWCVGKKIDFSLAEFALDYVGFITFNSENSESIFLSEI |
| | | QDTINIELLRSQVKQLKDQFKNLISRSSFGPIYRKDFENFICHAL |
| | | EEDRSQWLLDPIKINLSASSSQYQDLNLDISDFNGPDRAQKTSSD |
| | | WNSLIKKAVSIGDFIHNSGDRRTLLIDGKQRMSTACMLGYVFSAT |
| | | RNFLLEIEHNGLIYRTDDHKQKEGQFFTKIEAVEPQGETEAIVAI |
| | | GFPTAIGKDIDSTINEVKSLPRLNLESSHAIDNMETLNLAVREAK |
| | | SALVSFKSENKLSKLHLFIKAPSVFAMVLGHRLNGICDIQLYDWV |
| | | DGQYIPTAELNL |
| SEQ ID NO: 5 | Amino acid | MATSVLANWHGHDYQARYFWIEASRLKNPQQDFVVEVSYEADGPK |
| | sequence of | AFDDVITRYNPPRRSTGPDRIQADYYQIKFHVTQAASFGFEDLID |
| | EcCap4 wild | PAFIGAETFSILERLKQAKGTEPANSAFHLVTTDRIIDEDPLGEI |
| | sequence | ISNVDGSIRLDKLFDGTTDRSRKGKVRKLWRQHLKLSTDQELEQV |
| | | LSGFHIQQSQPTLEAMREKVNTCFQIIGLITCETSSDFRFDGAAR |
| | | ALRSQERYRFTREQFTALCEEENWIRSEAPESFRNVALRSFSDGP |
| | | LDIMDALPEHTLSLLSLFEGRFPSPGIEWNDVIKPQVETFLTGIR |
| | | QTERKVRLYLNTHSSIAMLAGKCLGHKSGVEIELVQKGRMGDSIW |
| | | SENESQDEPDAVIETETVGTGSDVAVVLSITRNALPKARAYILEN |
| | | QPDIGRIIHVTPANGHGQRSVKNGSHAVAIAEQVSDVVMDADLPV |
| | | EASLHIFSAAPNAVNFYLGQHTDFLGTCVFYEFDFQRQRDGSYLP |
| | | SFKV |
| SEQ ID NO: 6 | Amino Acid | MKKSIFDQFVNQYALSKTLRFELKPVGETGRMLEEAKVFAKDETI |
| | Sequence of | KKKYEATKPFFNKLHREFVEEALNEVELAGLPEYFEIFKYWKRYK |
| | Cas12p | KKFEKDLQKKEKELRKSVVGFFNAQAKEWAKKYETLGVKKKDVGL |
| | | LFEENVFAILKERYGNEEGSQIVDESTGKDVSIFDSWKGFTGYFI |
| | | KFQETRKNFYKDDGTATALATRIIDQNLKRFCDNLLIFESIRDKI |
| | | DFSEVEQTMGNSIDKVFSVIFYSSCLLQEGIDFYNCVLGGETLPN |
| | | GEKRQGINELINLYRQKTSEKVPFLKLLDKQILSEKEKFMDEIEN |
| | | DEALLDTLKIFRKSAEEKTTLLKNIFGDFVMNQGKYDLAQIYISR |
| | | ESLNTISRKWTSETDIFEDSLYEVLKKSKIVSASVKKKDGGYAFP |
| | | EFIALIYVKSALEQIPTEKFWKERYYKNIGDVLNKGFLNGKEGVW |
| | | LQFLLIFDFEFNSLFEREIIDENGDKKVAGYNLFAKGFDDLLNNF |
| | | KYDQKAKVVIKDFADEVLHIYQMGKYFAIEKKRSWLADYDIDSFY |
| | | TDPEKGYLKFYENAYEEIIQVYNKLRNYLTKKPYSEDKWKLNFEN |
| | | PTLADGWDKNKEADNSTVILKKDGRYYLGLMARGRNKLFDDRNLP |
| | | KILEGVENGKYEKVVYKYFPDQAKMFPKVCFSTKGLEFFQPSEEV |
| | | ITIYKNSEFKKGYTFNVRSMQRLIDFYKDCLVRYEGWQCYDFRNL |
| | | RKTEDYRKNIEEFFSDVAMDGYKISFQDVSESYIKEKNQNGDLYL |
| | | FEIKNKDWNEGANGKKNLHTIYFESLFSADNIAMNFPVKLNGQAE |
| | | IFYRPRTEGLEKERIITKKGNVLEKGDKAFHKRRYTENKVFFHVP |
| | | ITLNRTKKNPFQFNAKINDFLAKNSDINVIGVDRGEKQLAYFSVI |
| | | SQRGKILDRGSLNVINGVNYAEKLEEKARGREQARKDWQQIEGIK |
| | | DLKKGYISQVVRKLADLAIQYNAIIVFEDLNMRFKQIRGGIEKSV |
| | | YQQLEKALIDKLTFLVEKEEKDVEKAGHLLKAYQLAAPFETFQKM |
| | | GKQTGIVFYTQAAYTSRIDPVTGWRPHLYLKYSSAEKAKADLLKF |
| | | KKIKFVDGRFEFTYDIKSFREQKEHPKATVWTVCSCVERFRWNRY |
| | | LNSNKGGYDHYSDVTKFLVELFQEYGIDFERGDIVGQIEVLETKG |
| | | NEKFFKNFVFFFNLICQIRNTNASELAKKDGKDDFILSPVEPFFD |
| | | SRNSEKFGEDLPKNGDDNGAFNIARKGLVIMDKITKFADENGGCE |
| | | KMKWGDLYVSNVEWDNFVANK* |
| SEQ ID NO: 7 | Nucleic Acid | ATGAAAAAAAGCATTTTTGATCAGTTTGTGAACCAGTATGCGCTG |
| | Sequence of | AGCAAAACCCTGCGCTTCGAGCTGAAACCGGTGGGTGAAACCGGC |
| | Cas12p | CGTATGCTGGAGGAAGCGAAGGTTTTCGCGAAGGATGAAACCATT |
| | | AAGAAAAAGTACGAAGCGACCAAGCCGTTCTTTAACAAACTGCAC |
| | | CGTGAATTCGTGGAGGAAGCGCTGAACGAGGTTGAACTGGCGGGC |
| | | CTGCCGGAGTACTTCGAAATCTTCAAGTACTGGAAGCGTTACAAA |
| | | AAGAAATTCGAGAAGGACCTGCAGAAGAAAGAGAAGGAACTGCGT |
| | | AAAAGCGTGGTTGGTTTCTTTAACGCGCAAGCGAAGGAGTGGGCG |
| | | AAGAAATATGAAACCCTGGGCGTGAAGAAAAAGGATGTTGGTCTG |
| | | CTGTTCGAGGAAAACGTGTTTGCGATTCTGAAAGAACGTTACGGT |
| | | AACGAGGAAGGCAGCCAGATTGTGGACGAGAGCACCGGCAAGGAT |
| | | GTTAGCATCTTCGACAGCTGGAAGGGTTTTACCGGCTATTTCATC |
| | | AAATTTCAGGAAACCCGTAAGAACTTCTACAAAGATGATGGTACC |
| | | GCGACCGCGCTGGCGACCCGTATCATTGATCAAAACCTGAAACGT |
| | | TTCTGCGACAACCTGCTGATCTTTGAGAGCATTCGTGATAAGATC |
| | | GACTTCAGCGAGGTTGAACAGACCATGGGCAACAGCATCGATAAG |
| | | GTGTTCAGCGTTATCTTTTATAGCAGCTGCCTGCTGCAAGAAGGT |
| | | ATCGACTTTTACAACTGCGTGCTGGGTGGTGAAACCCTGCCGAAC |
| | | GGTGAAAAGCGTCAGGGCATTAACGAACTGATCAACCTGTACCGT |
| | | CAAAAGACCAGCGAGAAAGTTCCGTTCCTGAAGCTGCTGGACAAA |
| | | CAGATTCTGAGCGAGAAGGAAAAATTTATGGATGAGATCGAAAAC |
| | | GACGAGGCGCTGCTGGATACCCTGAAGATTTTCCGTAAAAGCGCG |
| | | GAGGAAAAGACCACCCTGCTGAAAAACATCTTCGGCGATTTTGTG |
| | | ATGAACCAGGGTAAATATGACCTGGCGCAAATCTACATTAGCCGT |
| | | GAAAGCCTGAACACCATTAGCCGTAAGTGGACCAGCGAAACCGAT |
| | | ATCTTCGAAGACAGCCTGTACGAGGTGCTGAAAAAGAGCAAAATC |
| | | GTGAGCGCGAGCGTTAAAAAGAAAGACGGTGGCTACGCGTTCCCG |
| | | GAGTTTATCGCGCTGATTTATGTTAAAAGCGCGCTGGAACAGATT |
| | | CCGACCGAGAAGTTCTGGAAAGAACGTTACTATAAGAACATCGGC |
| | | GATGTGCTGAACAAGGGTTTCCTGAACGGTAAAGAAGGCGTTTGG |
| | | CTGCAATTTCTGCTGATCTTTGACTTCGAATTTAACAGCCTGTTC |
| | | GAGCGTGAAATCATTGATGAGAACGGCGACAAGAAAGTGGCGGGT |
| | | TATAACCTGTTCGCGAAGGGTTTTGACGATCTGCTGAACAACTTC |
| | | AAATACGACCAGAAGGCGAAAGTGGTTATTAAGGATTTTGCGGAC |
| | | GAAGTTCTGCACATTTATCAAATGGGCAAATACTTCGCGATCGAG |
| | | AAGAAACGTAGCTGGCTGGCGGACTATGATATTGACAGCTTCTAC |
| | | ACCGATCCGGAGAAGGGTTACCTGAAATTTTATGAAAACGCGTAC |
| | | GAGGAAATCATTCAGGTTTATAACAAGCTGCGTAACTACCTGACC |
| | | AAGAAACCGTATAGCGAGGACAAGTGGAAACTGAACTTCGAAAAC |
| | | CCGACCCTGGCGGATGGTTGGGACAAGAACAAAGAGGCGGATAAC |
| | | AGCACCGTGATTCTGAAGAAAGACGGTCGTTACTATCTGGGCCTG |
| | | ATGGCGCGTGGTCGTAACAAGCTGTTCGACGATCGTAACCTGCCG |
| | | AAAATCCTGGAGGGTGTTGAAAACGGCAAGTACGAAAAGGTGGTT |
| | | TACAAGTACTTCCCGGATCAGGCGAAGATGTTCCCGAAAGTGTGC |
| | | TTTAGCACCAAAGGCCTGGAATTCTTTCAACCGAGCGAGGAAGTT |
| | | ATCACCATTTACAAGAACAGCGAGTTCAAGAAAGGTTATACCTTT |
| | | AACGTGCGTAGCATGCAGCGTCTGATTGATTTCTATAAAGACTGC |
| | | CTGGTTCGTTACGAAGGTTGGCAATGCTATGATTTTCGTAACCTG |
| | | CGTAAGACCGAGGACTACCGTAAAAACATCGAGGAATTCTTTAGC |
| | | GATGTGGCGATGGACGGCTACAAGATTAGCTTCCAGGACGTTAGC |
| | | GAGAGCTATATCAAGGAGAAGAACCAAAACGGTGATCTGTACCTG |
| | | TTTGAGATCAAGAACAAAGACTGGAACGAAGGTGCGAACGGCAAG |
| | | AAAAACCTGCACACCATTTATTTCGAGAGCCTGTTTAGCGCGGAT |
| | | AACATCGCGATGAACTTCCCGGTGAAACTGAACGGCCAGGCGGAG |
| | | ATCTTTTACCGTCCGCGTACCGAAGGTCTGGAGAAGGAACGTATC |
| | | ATTACCAAGAAAGGCAACGTTCTGGAAAAGGGTGACAAAGCGTTC |
| | | CACAAGCGTCGTTACACCGAGAACAAAGTGTTCTTTCACGTTCCG |
| | | ATTACCCTGAACCGTACCAAGAAAAACCCGTTCCAATTTAACGCG |
| | | AAGATCAACGACTTCCTGGCGAAAAACAGCGATATCAACGTGATT |
| | | GGTGTTGACCGTGGCGAGAAACAGCTGGCGTATTTTAGCGTGATT |
| | | AGCCAACGTGGCAAGATCCTGGACCGTGGTAGCCTGAACGTGATC |
| | | AACGGCGTTAACTACGCGGAGAAGCTGGAGGAAAAAGCGCGTGGT |
| | | CGTGAACAGGCGCGTAAGGATTGGCAGCAAATCGAGGGCATTAAA |
| | | GACCTGAAGAAAGGTTATATTAGCCAGGTGGTTCGTAAACTGGCG |
| | | GATCTGGCGATCCAATACAACGCGATCATTGTGTTCGAGGACCTG |
| | | AACATGCGTTTTAAGCAAATTCGTGGTGGCATCGAGAAAAGCGTT |
| | | TATCAGCAACTGGAAAAGGCGCTGATCGATAAACTGACCTTCCTG |
| | | GTGGAGAAGGAAGAAAAGGACGTTGAAAAGGCGGGTCACCTGCTG |
| | | AAAGCGTACCAGCTGGCGGCGCCGTTCGAAACCTTTCAGAAGATG |
| | | GGTAAACAAACCGGCATTGTGTTTTATACCCAAGCGGCGTACACC |
| | | AGCCGTATCGATCCGGTTACCGGCTGGCGTCCGCACCTGTACCTG |
| | | AAATATAGCAGCGCGGAAAAGGCGAAAGCGGACCTGCTGAAGTTC |
| | | AAGAAAATTAAGTTCGTGGATGGTCGTTTCGAGTTTACCTACGAC |
| | | ATCAAGAGCTTCCGTGAGCAGAAGGAACACCCGAAAGCGACCGTG |
| | | TGGACCGTTTGCAGCTGCGTTGAGCGTTTTCGTTGGAACCGTTAT |
| | | CTGAACAGCAACAAAGGTGGCTACGATCACTATAGCGACGTGACC |
| | | AAGTTCCTGGTTGAGCTGTTTCAGGAATACGGCATCGACTTCGAA |
| | | CGTGGTGATATTGTGGGCCAAATCGAGGTTCTGGAAACCAAGGGT |
| | | AACGAGAAGTTCTTTAAGAACTTCGTGTTCTTTTTCAACCTGATC |
| | | TGCCAGATTCGTAACACCAACGCGAGCGAACTGGCGAAGAAAGAC |
| | | GGCAAGGACGATTTCATTCTGAGCCCGGTTGAGCCGTTTTTCGAT |
| | | AGCCGTAACAGCGAGAAGTTCGGCGAAGACCTGCCGAAAAACGGT |
| | | GACGATAACGGCGCGTTTAACATCGCGCGTAAAGGTCTGGTTATT |
| | | ATGGATAAGATCACCAAATTCGCGGACGAGAACGGTGGCTGCGAA |
| | | AAGATGAAATGGGGTGACCTGTATGTGAGCAATGTGGAGTGGGAT |
| | | AACTTTGTGGCGAATAAATAA |

1. A method, comprising:
providing to a sample (i) a CRISPR/Cas complex comprising an effector nuclease and a guide RNA encoding a nucleic acid that hybridizes to a target nucleic acid, (ii) one or more nucleases, wherein the one or more nucleases is not the same as the effector nuclease, (iii) one or more oligonucleotides, and (iv) a fluorescence reporter, and
measuring a fluorescence signal emitted from the fluorescence reporter, wherein a presence of the fluorescence signal indicates the presence of the target nucleic acid in the sample.

2. A The method of claim 1,
wherein when the target nucleic acid sequence is present in the sample, the CRISPR/Cas complex cleaves the target nucleic acid and the CRISPR/Cas complex exhibits collateral cleavage activity and cleaves the one or more oligonucleotides;
wherein the one or more oligonucleotides activates the one or more nucleases; and
wherein the one or more additional nuclease cleaves the fluorescence reporter to amplify the fluorescence.

3-6. (canceled)

7. The method of claim 1, wherein the one or more nucleases is an unspecific nuclease.

8. The method of claim 7, wherein the unspecific nuclease is Csx1, Cap4, Can1, NucC, or combinations thereof.

9. The method of claim 1, wherein the effector nuclease is a Cas12 protein or a Cas13 protein.

10. (canceled)

11. The method of claim 9, wherein the effector nuclease is a Cas12p protein comprising SEQ ID NO: 6.

12-18. (canceled)

19. The method of claim 1, wherein the one or more oligonucleotides is a cyclic oligonucleotide, a linear oligonucleotide, a polynucleotide, or combinations thereof.

20. The method of claim 1, wherein the guide RNA is designed to detect a single nucleotide polymorphism in a target nucleic acid or a splice variant of an RNA transcript.

21. The method of claim 1, wherein the fluorescence reporter is a FAM-Q reporter.

22-40. (canceled)

41. A kit, comprising: (i) a CRISPR/Cas complex comprising an effector nuclease and a guide RNA encoding a nucleic acid that hybridizes to a target nucleic acid, (ii) one or more nucleases, wherein the one or more nucleases is not the same as the effector nuclease, (iii) one or more oligonucleotides, and (iv) a fluorescence reporter, wherein the effector nuclease exhibits collateral nucleic acid cleavage activity.

42-76. (canceled)

77. A method for identifying a subject having a disease, the method comprising:
providing to a sample (i) a CRISPR/Cas complex comprising an effector nuclease and a guide RNA encoding a nucleic acid that hybridizes to a target nucleic acid, (ii) one or more nucleases, wherein the one or more nucleases is not the same as the effector nuclease, (iii) one or more oligonucleotides, and (iv) a fluorescence reporter, wherein the effector nuclease exhibits collateral nucleic acid cleavage activity, and
measuring a fluorescence signal emitted from the fluorescence reporter, wherein the presence of the fluorescence signal indicates the presence of disease.

78-93. (canceled)

94. The method of claim 77, wherein the target nucleic acid is a viral nucleic acid, a bacterial nucleic acid, a fungal nucleic acid, a nucleic acid from a parasite, or a nucleic acid from a protozoa.

95. The method of claim 94, wherein the target nucleic acid is the viral nucleic acid, and the viral nucleic acid is from a double stranded RNA virus, a positive sense RNA virus, a negative sense RNA virus, a retrovirus, or combinations thereof.

96. The method of claim 95, wherein the viral nucleic acid is from a Myoviridae, a Podoviridae, a Siphoviridae, an Alloherpesviridae, a Herpesviridae, a Malocoherpesviridae, a Lipothrixviridae, a Rudiviridae, an Adenoviridae, an Ampullaviridae, an Ascoviridae, an Asfarviridae, a Baculoviridae, a Cicaudaviridae, a Clavaviridae, a Corticoviridae, a Fuselloviridae, a Globuloviridae, a Guttaviridae, a Hytrosaviridae, a Iridoviridae, a Maseilleviridae, a Mimiviridae, a Nudiviridae, a Nimaviridae, a Pandoraviridae, a Papillomaviridae, a Phycodnaviridae, a Plasmaviridae, a Polydnaviruses, a Polyomaviridae, a Poxviridae, a Sphaerolipoviridae, a Tectiviridae, a Turriviridae, a Dinodnavirus, a Salterprovirus, a Rhizidovirus, a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus

97. (canceled)

98. The method of claim 94, wherein the target nucleic acid is the bacterial nucleic acid, and the bacterial nucleic acid is from an Acinetobacter, an Actinobacillus, an Actinomycete, an Actinomyces, an Aerococcus, an Aeromonas, an Anaplasma, an Alcaligenes, a Bacillus, a Bacteroides, a Bartonella, a Bifidobacterium, a Bordetella, a Borrelia, a Brucella, a Burkholderia, a Campylobacter, a Capnocytophaga, a Chlamydia, a Citrobacter, a Coxiella, a Corynbacterium, a Clostridium, an Eikenella, an Enterobacter, an Escherichia, an Enterococcus, an Ehlichia, an Epidermophyton, an Erysipelothrix, a Eubacterium, a Francisella, a Fusobacterium, a Gardnerella, a Gemella, a Haemophilus, a Helicobacter, a Kingella, a Klebsiella, a Lactobacillus, a Lactococcus, a Listeria, a Leptospira, a Legionella, a Leptospira, Leuconostoc, a Mannheimia, a Microsporum, a Micrococcus, a Moraxella, a Morganell, a Mobiluncus, a Micrococcus, Mycobacterium, a Mycoplasm, a Nocardia, a Neisseria, a Pasteurelaa, a Pediococcus, a Peptostreptococcus, a Pityrosporum, a Plesiomonas, a Prevotella, a Porphyromonas, a Proteus, a Providencia, a Pseudomonas, a Propionibacteriums, a Rhodococcus, a Rickettsia, a Rhodococcus, a Serratia, a Stenotrophomonas, a Salmonella, a Serratia, a Shigella, a Staphylococcus, a Streptococcus, a Spirillum, a Streptobacillus, a Treponema, a Tropheryma, a Trichophyton, a Ureaplasma, a Veillonella, a Vibrio, a Yersinia, a Xanthomonas, or combinations thereof

99. (canceled)

100. The method of claim 94, wherein the target nucleic acid is the fungal nucleic acid, and the fungal nucleic acid is from Aspergillus, Blastomyces, Candidiasis, Coccidiodomycosis, Cryptococcus neqformans, Cryptococcus gatti, sp. Histoplasma, Pneumocystis sp., Stachybotrys, Mucroymcosis, Sporothrix, Exserohilum, Cladosporium, Geotrichum, Saccharomyces, Hansenula, Candida, Kluyveromyces, Debaryomyces, Pichia, Penicillium, Cladosporium, Byssochlamys or a combination thereof.

101. (canceled)

102. The method of claim 94, wherein the target nucleic acid is the nucleic acid from a parasite, and the parasite is Trypanosoma cruzi, T. brucei gambiense, T. brucei rhodesiense, Leishmania braziliensis, L. infantum, L. mexicana, L. major, L. tropica, L. donovani, Naegleria fowleri, Giardia intestinalis (G. lamblia, G. duodenalis), canthamoeba castellanii, Balamuthia madrillaris, Entamoeba histolytica, Blastocystic hominis, Babesia microti, Cryptosporidium parvum, Cyclospora cayetanensis, Plasmodium falciparum, P. vivax, P. ovale, P. malariae, and Toxoplasma gondii, or combinations thereof.

103. (canceled)

104. The method of claim 94, wherein the target nucleic acid is the nucleic acid from a protozoa, and the protozoa is a Euglenozoa, a Heterolobosea, a Diplomonadida, an Amoebozoa, a Blastocystic, an Apicomplexa, or combinations thereof.

105. The method of claim 77, wherein the disease is cancer, an autoimmune disease, or an infection.

106. The method of claim 105, wherein the infection is caused by a virus, a bacterium, a fungus, a protozoa, or a parasite.

107. The method of claim 106, wherein the viral infection is caused by Coronavirus, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

108. The method of claim 77, wherein the sample is blood, plasma, serum, saliva, urine, stool, sputum, mucous, a tissue biopsy, lymph fluid, synovial fluid, bile, ascites, pleural effusion, seroma, saliva, cerebrospinal fluid, aqueous or vitreous humor, any bodily secretion, a transudate, an exudate, fluid obtained from a joint, or a swab of skin or mucosal membrane surface.

109. The method of claim, wherein the target nucleic acid is the viral nucleic acid, and the viral nucleic acid is from severe acute respiratory syndrome coronavirus 2 (SARS-CoV-2) or influenza.


SEQUENCE LISTING (22 sequences)

SEQ ID NO: 1 - protein, 472 aa, Sulfolobus islandicus
AAHHHHHHSSGLVPRGSHMKCLFYIAGDVSNYSIVNYELNGQTQNTFFAAHALYNLFKPDKVIALIPDSLVKDNVSDEECYKNLVINRAKELNFAGMEEFMNKVEIRKIPNVGIASAIQCENGAPKKEKNKEGREVLKRLPYNEKRSPIFIFNAIYAIFKDEACDEYLVDLTHGTNVLVSIGMNVGALFNAKFYSAPVMGMPGKDSIVNIVELTDVVQATNDSLMIRSSIENLDERYFKDYSAKLSRLNPTIFEEEEKKVLTRVKGTDVNVVINFLWNIRNGFTVNAVKSMNELKNIINQLEEDLEKLKSFYKNWEEHKNFQGETLLVLSDLDSTLKVKDLLIEGNDLEKLNYLLDLYIKASIYDKALSLARELPVAICLNKVGGGMFDDKNEKYKHCNEIVTSYLRLRYSGLMEFRNTLMHGGLSTDMKPNVDKDGNITPGKIVTKNKIEDFVKRELRNYFDKIVNFLSSA

SEQ ID NO: 2 - protein, 241 aa, Escherichia coli
MSDWSLSQLFASLHEDIQLRLGTARKAFQHPGAKGDASEGVWIEMLDTYLPKRYQAANAFVVDSLGNFSDQIDVVVFDRQYSPFIFKFNEQIIVPAESVYAVFEAKQSASADLVAYAQRKVASVRRLHRTSLPIPHAGGTYPAKPLIPILGGLLTFESDWSPALGMSFDKALNGDLSDGRLDMGCVASHGHFYFNNIDSKFNFEHGNKPATAFLFRLIAQLQFSGTVPMIDIDAYGKWLAN

SEQ ID NO: 3 - protein, 636 aa, Thermus thermophilus
MQAPVYLCLLGNDPAPAYLGLKVVEREAGRVAKAVFYSFPAWNEEYGKKRQAFFRLLSEKGVLYEERPLEKGLEEAEAREVWVNLTGGAKYWAVRFLGHWRRPGARVFLVEGHRALEAPRALFLWPREEERSLEAEALTLEEYARLYLEPLGEAWERVSPPGAFPPGAQAARLPGREGGVFVVHRGLPYWYWVRPHLGGEAKDMSRKALSAFSGEAKRLGGQLCLPVVPYHKAHLRSRHPKERENVFARWRAWAREYGVFLVDPGRPLEEEVASLIKGKASKKALPLPQEGPLLLALVSEQAVPLYAAYLHAGPREVYLLTTPEMESRLRWAEAFFRGKGVRVHRSFLSGPWALREVRDLLAPVVEEALRRGHPVHANLNSGTTAMALGLYLALRDGARAHYLDGDRLLLLDGGEAEVPWEEGRPEDLLALRGYRFEEEYPDARPDPGLLALAEEILRRWDEVQTSWEASPLVRRFLKFWKKRFGQAFPPKRLSRLKGLPLEYAVYSHLNAHLAPKGGQARMGGHLVPLGGNEALAPQSTEVDGVFFHRGALWFVECKPTDEGLRERAPIMAELVRSVGGVEARGLMVARRWRGAPPPASPNLVYMALEGGEGVGVYRFPEELEKALSRNPAPRRG

SEQ ID NO: 4 - protein, 462 aa, Acinetobacter baumannii
MSASLLEKQSTGGAIARVGFGYQDAFVLRSLPLWLSQSAFSHIVSEALSDIEVCYFSSEKSLHVMYEAKNHSLTATEFWDEIRRFKSLFDTHPKNFIWFNLVCPSYNTAISPLISKIDRLRGVGSSYDDDSSVSVNGRSEYLDWCVGKKIDFSLAEFALDYVGFITFNSENSESIFLSEIQDTINIELLRSQVKQLKDQFKNLISRSSFGPIYRKDFENFICHALEEDRSQWLLDPIKINLSASSSQYQDLNLDISDFNGPDRAQKTSSDWNSLIKKAVSIGDFIHNSGDRRTLLIDGKQRMSTACMLGYVFSATRNFLLEIEHNGLIYRTDDHKQKEGQFFTKIEAVEPQGETEAIVAIGFPTAIGKDIDSTINEVKSLPRLNLESSHAIDNMETLNLAVREAKSALVSFKSENKLSKLHLFIKAPSVFAMVLGHRLNGICDIQLYDWVDGQYIPTAELNL

SEQ ID NO: 5 - protein, 499 aa, Enterobacter cloacae
MATSVLANWHGHDYQARYFWIEASRLKNPQQDFVVEVSYEADGPKAFDDVITRYNPPRRSTGPDRIQADYYQIKFHVTQAASFGFEDLIDPAFIGAETFSILERLKQAKGTEPANSAFHLVTTDRIIDEDPLGEIISNVDGSIRLDKLFDGTTDRSRKGKVRKLWRQHLKLSTDQELEQVLSGFHIQQSQPTLEAMREKVNTCFQIIGLITCETSSDFRFDGAARALRSQERYRFTREQFTALCEEENWIRSEAPESFRNVALRSFSDGPLDIMDALPEHTLSLLSLFEGRFPSPGIEWNDVIKPQVETFLTGIRQTERKVRLYLNTHSSIAMLAGKCLGHKSGVEIELVQKGRMGDSIWSENESQDEPDAVIETETVGTGSDVAVVLSITRNALPKARAYILENQPDIGRIIHVTPANGHGQRSVKNGSHAVAIAEQVSDVVMDADLPVEASLHIFSAAPNAVNFYLGQHTDFLGTCVFYEFDFQRQRDGSYLPSFKV

SEQ ID NO: 6 - protein, 1281 aa, Artificial Sequence
  Description of Artificial Sequence Synthetic polypeptide
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 7 - DNA, 3846 bp, Artificial Sequence
  Description of Artificial Sequence Synthetic polynucleotide
atgaaaaaaagcatttttgatcagtttgtgaaccagtatgcgctgagcaaaaccctgcgcttcgagctgaaaccggtgggtgaaaccggccgtatgctggaggaagcgaaggttttcgcgaaggatgaaaccattaagaaaaagtacgaagcgaccaagccgttctttaacaaactgcaccgtgaattcgtggaggaagcgctgaacgaggttgaactggcgggcctgccggagtacttcgaaatcttcaagtactggaagcgttacaaaaagaaattcgagaaggacctgcagaagaaagagaaggaactgcgtaaaagcgtggttggtttctttaacgcgcaagcgaaggagtgggcgaagaaatatgaaaccctgggcgtgaagaaaaaggatgttggtctgctgttcgaggaaaacgtgtttgcgattctgaaagaacgttacggtaacgaggaaggcagccagattgtggacgagagcaccggcaaggatgttagcatcttcgacagctggaagggttttaccggctatttcatcaaatttcaggaaacccgtaagaacttctacaaagatgatggtaccgcgaccgcgctggcgacccgtatcattgatcaaaacctgaaacgtttctgcgacaacctgctgatctttgagagcattcgtgataagatcgacttcagcgaggttgaacagaccatgggcaacagcatcgataaggtgttcagcgttatcttttatagcagctgcctgctgcaagaaggtatcgacttttacaactgcgtgctgggtggtgaaaccctgccgaacggtgaaaagcgtcagggcattaacgaactgatcaacctgtaccgtcaaaagaccagcgagaaagttccgttcctgaagctgctggacaaacagattctgagcgagaaggaaaaatttatggatgagatcgaaaacgacgaggcgctgctggataccctgaagattttccgtaaaagcgcggaggaaaagaccaccctgctgaaaaacatcttcggcgattttgtgatgaaccagggtaaatatgacctggcgcaaatctacattagccgtgaaagcctgaacaccattagccgtaagtggaccagcgaaaccgatatcttcgaagacagcctgtacgaggtgctgaaaaagagcaaaatcgtgagcgcgagcgttaaaaagaaagacggtggctacgcgttcccggagtttatcgcgctgatttatgttaaaagcgcgctggaacagattccgaccgagaagttctggaaagaacgttactataagaacatcggcgatgtgctgaacaagggtttcctgaacggtaaagaaggcgtttggctgcaatttctgctgatctttgacttcgaatttaacagcctgttcgagcgtgaaatcattgatgagaacggcgacaagaaagtggcgggttataacctgttcgcgaagggttttgacgatctgctgaacaacttcaaatacgaccagaaggcgaaagtggttattaaggattttgcggacgaagttctgcacatttatcaaatgggcaaatacttcgcgatcgagaagaaacgtagctggctggcggactatgatattgacagcttctacaccgatccggagaagggttacctgaaattttatgaaaacgcgtacgaggaaatcattcaggtttataacaagctgcgtaactacctgaccaagaaaccgtatagcgaggacaagtggaaactgaacttcgaaaacccgaccctggcggatggttgggacaagaacaaagaggcggataacagcaccgtgattctgaagaaagacggtcgttactatctgggcctgatggcgcgtggtcgtaacaagctgttcgacgatcgtaacctgccgaaaatcctggagggtgttgaaaacggcaagtacgaaaaggtggtttacaagtacttcccggatcaggcgaagatgttcccgaaagtgtgctttagcaccaaaggcctggaattctttcaaccgagcgaggaagttatcaccatttacaagaacagcgagttcaagaaaggttatacctttaacgtgcgtagcatgcagcgtctgattgatttctataaagactgcctggttcgttacgaaggttggcaatgctatgattttcgtaacctgcgtaagaccgaggactaccgtaaaaacatcgaggaattctttagcgatgtggcgatggacggctacaagattagcttccaggacgttagcgagagctatatcaaggagaagaaccaaaacggtgatctgtacctgtttgagatcaagaacaaagactggaacgaaggtgcgaacggcaagaaaaacctgcacaccatttatttcgagagcctgtttagcgcggataacatcgcgatgaacttcccggtgaaactgaacggccaggcggagatcttttaccgtccgcgtaccgaaggtctggagaaggaacgtatcattaccaagaaaggcaacgttctggaaaagggtgacaaagcgttccacaagcgtcgttacaccgagaacaaagtgttctttcacgttccgattaccctgaaccgtaccaagaaaaacccgttccaatttaacgcgaagatcaacgacttcctggcgaaaaacagcgatatcaacgtgattggtgttgaccgtggcgagaaacagctggcgtattttagcgtgattagccaacgtggcaagatcctggaccgtggtagcctgaacgtgatcaacggcgttaactacgcggagaagctggaggaaaaagcgcgtggtcgtgaacaggcgcgtaaggattggcagcaaatcgagggcattaaagacctgaagaaaggttatattagccaggtggttcgtaaactggcggatctggcgatccaatacaacgcgatcattgtgttcgaggacctgaacatgcgttttaagcaaattcgtggtggcatcgagaaaagcgtttatcagcaactggaaaaggcgctgatcgataaactgaccttcctggtggagaaggaagaaaaggacgttgaaaaggcgggtcacctgctgaaagcgtaccagctggcggcgccgttcgaaacctttcagaagatgggtaaacaaaccggcattgtgttttatacccaagcggcgtacaccagccgtatcgatccggttaccggctggcgtccgcacctgtacctgaaatatagcagcgcggaaaaggcgaaagcggacctgctgaagttcaagaaaattaagttcgtggatggtcgtttcgagtttacctacgacatcaagagcttccgtgagcagaaggaacacccgaaagcgaccgtgtggaccgtttgcagctgcgttgagcgttttcgttggaaccgttatctgaacagcaacaaaggtggctacgatcactatagcgacgtgaccaagttcctggttgagctgtttcaggaatacggcatcgacttcgaacgtggtgatattgtgggccaaatcgaggttctggaaaccaagggtaacgagaagttctttaagaacttcgtgttctttttcaacctgatctgccagattcgtaacaccaacgcgagcgaactggcgaagaaagacggcaaggacgatttcattctgagcccggttgagccgtttttcgatagccgtaacagcgagaagttcggcgaagacctgccgaaaaacggtgacgataacggcgcgtttaacatcgcgcgtaaaggtctggttattatggataagatcaccaaattcgcggacgagaacggtggctgcgaaaagatgaaatggggtgacctgtatgtgagcaatgtggagtgggataactttgtggcgaataaataa

SEQ ID NO: 8 - protein, 9 aa, Unknown
  Description of Unknown “LAGLIDADG” motif sequence
LAGLIDADG

SEQ ID NO: 9 - RNA, 18 nt, Artificial Sequence
  Description of Artificial Sequence Synthetic oligonucleotide
cccccccccccccccccc

SEQ ID NO: 10 - RNA, 19 nt, Artificial Sequence
  Description of Artificial Sequence Synthetic oligonucleotide
ccccccccccccccccccu

SEQ ID NO: 11 - RNA, 20 nt, Artificial Sequence
  Description of Artificial Sequence Synthetic oligonucleotide
ccccccccccccccccccuu

SEQ ID NO: 12 - RNA, 21 nt, Artificial Sequence
  Description of Artificial Sequence Synthetic oligonucleotide
ccccccccccccccccccuua

SEQ ID NO: 13 - RNA, 22 nt, Artificial Sequence
  Description of Artificial Sequence Synthetic oligonucleotide
ccccccccccccccccccuuau

SEQ ID NO: 14 - RNA, 23 nt, Artificial Sequence
  Description of Artificial Sequence Synthetic oligonucleotide
ccccccccccccccccccuuauu

SEQ ID NO: 15 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 16 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 17 - protein, 425 aa, Unknown
  Description of Unknown Unclassified Synechocystis sequence
MRTAITKIISFLGFNSYEVTTYKHPTQSIHLTTKYYQEALAEFYQPQKIYIFLTPTVETKCPGRGDQKEDRSNWHQLEEILAQKFPDIEIVPVKNIPERNSPEDIWTIFNKVNECLEDGDRVIFDITHSFRSVPIVALLAVSYFRVVRNITIEGLLYGAFEAKENGITPTFDLLPVVRLFDWLTATDQFLKTGNGEDLAKLLQEAEGHTQALGDRILEISQGLQLLRPMDVMTAAAALPESIEQGKPDLARVVPPFELLLNRVVSEYGQFALAEPKNYQVNGKAALQKQFFEIEWYAKKGLYVQAISLGREWLPSLLCYNFELDPMDKQAREEMEILCCGGKVGERESIYRSQYKEIEESKRKMINQLWNNPPCKLANLRNDVLHSGFRKDPKSVEDIKQTLQKIVEDLKKIAQVWGVFDEEYKS

SEQ ID NO: 18 - protein, 480 aa, Pyrococcus furiosus
MGMRVLVTTWGNPFQWEPITYEYRGIKVKSRNTLPILVKTLEPERILILVADTMANYYDSGKNKPEIEEKSFSSYSEVVEDTKERILWHIKEEVIEELREEDPELAKKIENMLKDERITIEVLPGVGVFGNITVEGEMLDFYYYATYKLAEWLPVQNNLEVYLDLTHGINFMPTFTYRALRNLLGLLAYLYNVKFEIVNSEPYPLGVSQEIREDTILHIREIGEGVVRPRPQYSPVEGKLYWNAFISSVANGFPLVFASFYPNIRDVEDYLNKKLEEFLVGIEVGEREDGKPYVKREKALDRSFKNASKLYYALRVFNTKFQNYPKKEVPIEEIMEISKIFESLPRIGIILERQVEWLRNLVYGRLWYENGEQKIKKGLLEIIKDKKDKRKEAEALKKGKTISLAEAAKLTRIFSPSGERIETIESPNVVRNFIAHSGFEYNIVYVKYDRLSDRLYFFYKDKEKAANLAYEALLYRGEKE

SEQ ID NO: 19 - protein, 464 aa, Thermus thermophilus
MEDLDALWERYREAVRARGNPQALYQEMVWPALLALWREKPRVYPFPQAFAVSVHTLGTSPEATALAILGAGAERVYVLHTPESARFLPRLRQDTGKDLYPVEIGKSDVEAIYREVKRLLEKHPEVPVALDLTSGTKAMSAGLAAAGFFFQRFYPKVRVVYVDNEDYDPELRRPRAGTEKLRILPNPHEALAEVDALFAKELYGKGEFGQAAAYFRGMVGRTGNQAYALYALLAEMYRAWRALDFGEALKAGRKLLGQLSQNVWLNHPLNARREALEAQVALLEAVDRFLKARDFALKEGVYGLARTLLHLAQEAKEEAAVLAALYAYRALELLLQERLALLGRRAEAPGLSPEEAEALRKALAELLGVLPEEVRLPAKLGLLDLLAFLRLKGDEALGRLSLAELRGLAGALKGRNSALLVHGFDVPSPKAVEGIARLAQGLLQDLEARTALGPLSPEPVPLGF

SEQ ID NO: 20 - protein, 241 aa, Escherichia coli; identical to SEQ ID NO: 2

SEQ ID NO: 21 - protein, 241 aa, Pseudomona aeruginosa
MSQWSLSQLLSSLHEDIQQRLSVVRKTFGHPGTKGDASENVWIDMLDTYLPKRYQAAKAHVVDSLGNFSQQIDVVVFDRQYSPFIFTYENETIIPAESVYAVFEAKQTADAGLVAYAQEKVASVRRLHRTSLPIPHAGGTYPAKPLIPILGGLLTFESEWSPALGPSMDKALNANLTEGRLDIGCVAAHGHFFYDQASGAYSYTNENKPATAFLFKLIAQLQFSGTVPMIDVEAYGQWLTK

SEQ ID NO: 22 - RNA, 12 nt, Artificial Sequence
  Description of Artificial Sequence Synthetic oligonucleotide
auguccccugaa